(19) United States
(12) Patent Application Publication
(10) Pub. No.: US 2010/0304480 A1
(43) Pub. Date: Dec. 2, 2010

(54) Hematopoietic Fingerprints: Methods of Use

(76) Inventors: Margaret A. Goodell, Houston, TX (US); Stuart M. Chambers, Houston, TX (US); Nathan C. Boles, Houston, TX (US); Kuan-yin K. Lin, Houston, TX (US); Megan P. Tierney, Houston, TX (US)

Correspondence Address: DRINKER BIDDLE & REATH;ATTN: INTELLECTUAL PROPERTY GROUP, ONE LOGAN SQUARE, SUITE 2000, PHILADELPHIA, PA, 19103-6996, US

(21) Appl. No.: 12/738,255

(22) PCT Filed: Oct. 17, 2008
(86) PCT No.: PCT/US08/80324

§ 371 Date: Aug. 10, 2010

Related U.S. Application Data
(60) Provisional application No. 60/999,595, filed on Oct. 19, 2007.

(51) Int. Cl. C12N 5/0789

(52) U.S. Cl. 435/366; 435/377

(57) ABSTRACT

The present invention is related to the discovery that the detection of one or more biomarkers in a body sample can identify hematapoietic progenitors that are precursors to a specific blood cell lineage. The methods of the present invention are also directed to the detection of the dysregulation of these biomarkers as a diagnostic assay for the certain disease states, such as cancer.

### CROSS-REFERENCE TO RELATED APPLICATIONS

This is a National Stage application of PCT International Application No. PCT/US2008/080324, filed Oct. 17, 2008, which in turn claims the benefit pursuant to 35 U.S.C. §119(e) of U.S. Provisional Application No. 60/999,595 filed on Oct. 19, 2007 which are hereby incorporated by reference in their entirety herein.

### STATEMENT REGARDING FEDERALLY SPONSORED RESEARCH OR DEVELOPMENT

This invention was made with government support under grant numbers T32-AI07495, T32-AG000183, DK63588 and DK58192, awarded by the National Institutes of Health. The government has certain rights in the invention.

### BACKGROUND OF THE INVENTION

Hematopoiesis is defined by stepwise differentiation of hematopoietic stem cells (HSC), through progenitor intermediates, to terminally differentiated blood cells with vastly different morphologies and functions. Regulation of this process is complex, involving association of HSC with their specialized niche, signals from stromal cells, epigenetic silencing of genetic programs specifying alternative lineage fates, and crosstalk between HSC and non-stem cell neighbors. The transcriptional control of HSC differentiation is still poorly understood, despite advances in mouse genetic techniques that have elucidated the role of certain pivotal molecules within the developmental hierarchy. A few transcription factors have been shown to be essential for specific lineages; for example, Early B-cell factor-1 (ebf1) in B lymphocytes (Lin and Grosschedl, 1995, Nature 376:263-267), and gata2 in megakaryocytic differentiation (Orkin et al., 1998, Stem Cells 16:79-83; Ling et al., 2004, J. Exp. Med. 200:871-882). The roles of these and other genes were identified empirically, and thus the number of genes demonstrated to be critical for differentiation within each hematopoietic population is extremely small or non-existent. The few global approaches that have been used to study regulation of hematopoietic cells have focused either on comparisons between HSC and other stem cell types (Ivanova et al., 2002, Science 298:601-604; Ramalho et al., 2002, Science, 298:597-600), or between HSC and pools of their differentiated progeny (Toren et al., 2005, Stem Cells 23:1142-1153). The drawback of these approaches is that many important regulatory candidates will not be identified because of lack of purity in the comparator populations.

There exists in the art an urgent need to be able to direct hematopoietic stem cell and hematopoietic progenitor cell differentiation to a selected terminally differentiated blood cell. The present invention answers this need.

### SUMMARY OF THE INVENTION

One embodiment of the invention comprises a method of directing the differentiation of a hematopoietic stem cell (HSC), a hematopoietic progenitor cell (HPC), or a combination thereof, said method comprising introducing into said cell a zinc finger protein (Zfp105), wherein said Zfp105 upregulates expression of a natural killer (NK) cell-specific polynucleotide in said cell, thereby inducing differentiation of said cell into an NK cell. In one aspect of the invention, the Zfp105 is delivered to said cell as a polypeptide. In another aspect of the invention, the Zfp105 is delivered to said cell as a nucleic acid. In another aspect of the invention, the nucleic acid is contained within a vector. In another aspect of the invention, the vector is a viral vector. In another aspect of the invention, the viral vector is selected from the group consisting of a retroviral vector, an adenoviral vector, and an adeno-associated viral vector. In another aspect of the invention, the vector is a non-viral vector. In another aspect, the cell is a human cell.

Another embodiment of the invention comprises a method of directing the differentiation of a HSC, a HPC, or a combination thereof, said method comprising introducing into said cell an Ets2, wherein said Ets2 upregulates expression of a monocyte-specific polynucleotide in said cell thereby inducing differentiation of said cell into a monocyte. In one aspect of the invention, the Ets2 is delivered to said cell as a polypeptide. In another aspect of the invention, the Ets2 is delivered to said cell as a nucleic acid. In another aspect of the invention, the nucleic acid is contained within a vector. In another aspect of the invention, the vector is a viral vector. In another aspect of the invention, the viral vector is selected from the group consisting of a retroviral vector, an adenoviral vector, and an adeno-associated viral vector. In another aspect of the invention, the vector is a non-viral vector. In another aspect, the cell is a human cell.

Yet another embodiment of the invention comprises a method of directing the differentiation of a HSC, a HPC, or a combination thereof, to a B-cell, said method comprising introducing to said cell at least one biomarker selected from the list consisting of Chd7 (chromodomain helicase DNA binding protein 7), Edaradd (EDAR (ectodysplasin-A receptor)-associated death domain, 2210016F16Rik, Dzip1 (DAZ interacting protein 1), and Tbl1x (transducin (beta)-like 1 X-linked). In one aspect of the invention, the biomarker is delivered to said cell as a polypeptide. In another aspect of the invention, the biomarker is delivered to said cell as a nucleic acid. In another aspect of the invention, the nucleic acid is contained within a vector. In another aspect of the invention, the vector is a viral vector. In another aspect of the invention, the viral vector is selected from the group consisting of a retroviral vector, an adenoviral vector, and an adeno-associated viral vector. In another aspect of the invention, the vector is a non-viral vector. In another aspect, the cell is a human cell.

Still another embodiment of the invention comprises a method of directing the differentiation of a HSC, a HPC, or a combination thereof, to a cell of the myeloid lineage, said method comprising introducing to said cell at least one biomarker selected from the list consisting of Med8 (mediator of RNA polymerase II transcription, subunit 8 homolog), Med14 (mediator complex subunit 14), GLIS2 (GLIS family zinc finger 2), Tnfaip8l1 (tumor necrosis factor, alpha-induced protein 8-like 1), and Mina (myc induced nuclear antigen). In one aspect of the invention, the biomarker is delivered to said cell as a polypeptide. In another aspect of the invention, the biomarker is delivered to said cell as a nucleic acid. In another aspect of the invention, the nucleic acid is contained within a vector. In another aspect of the invention, the vector is a viral vector. In another aspect of the invention, the viral vector is selected from the group consisting of a retroviral vector, an adenoviral vector, and an adeno-associated viral vector. In another aspect of the invention, the vector is a non-viral vector. In another aspect, the cell is a human cell.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIG. 1A and FIG. 1B, is a series of charts that illustrate how global transcription profile analysis reveals hematopoietic cell ontogeny. FIG. 1A depicts a dendrogram where the right-most branch point indicates the cell types with the highest degree of similarity. FIG. 1B depicts relative distance is collapsed to two dimensions using PCA, the HSC resides at a midpoint between lymphocytes, myeloid cells, and erythrocytes.

FIG. 2, comprising FIG. 2A through FIG. 2D, is a series of charts depicting unique genetic fingerprints for hematopoietic cell types. FIG. 2A depicts three examples of genes expressed in a specific cell type. The normalized (log2) expression intensity for each cell type for six example genes is shown. The grey line indicates the threshold window, above which a gene was considered to be expressed, and below which, non-expressed. FIG. 2B depicts gene expression in all differentiated cells, lymphocytes, or myeloid cells using a similar analysis. FIG. 2C is a heat map summarizing finger print pattern. All the single cell type fingerprint patterns are summarized in the heat map where the color intensity indicates normalized level of expression (log2) with darker shades corresponding to higher expression. FIG. 2D depicts the shared fingerprint patterns.

FIG. 3, comprising FIG. 3A through FIG. 3C, is a series of charts depicting examples of fingerprint genes and phenotype assessment. FIG. 3A depicts selected genes from each of the cell-type-specific and shared fingerprints in relation to their developmental relationships. FIG. 3B depicts a Venn diagram which summarizes the number of shared genes in the different fingerprints. FIG. 3C depicts a Venn diagram that summarizes the number of knockout mice with hematopoietic phenotypes in each of the indicated gene groups. For clarity, not all cell types and intersections have been illustrated.

FIG. 4, comprising FIG. 4A through FIG. 4D, is a series of charts which illustrate that T-cell and HSC activation share a similar transcriptional repertoire. FIG. 4A depicts the results of a cluster analysis. FIG. 4B depicts a pair-wise comparison between naïve (bottom panel) and activated T-cells (top panel, including both CD4+ and CD8+ cells) used to generate a list of genes up regulated in activated or naive T-cells. FIG. 4C depicts shared fingerprints for CD4+, CD8+, and ‘naïve’ and ‘activated T-cells. FIG. 4D depicts shared fingerprints for CD4+, CD8+, and ‘naïve’ and ‘activated T-cells. Gene list sizes range from 4 to 215, and are indicated to the right side of each heat map.

FIG. 5 is a graph which depicts the molecular pathway analysis of hematopoietic cells using KEGG to identify which cell types express an over- or under-abundance of components of a molecular pathways, with significant differences between cell types determined by an ANOVA (one way, a=0.05). For pathways containing genes found on the array, the mean expression value for all genes within a pathway for each cell type is displayed as a function of color density, where light coloration corresponds to a low average expression and dark coloration to a high average expression. Pathways are ordered by high expression within a cell type from left to right. Below the heat map, the number of neutral to over-abundant categories is denoted, and the percent of those pathways that are signaling and metabolic is indicated. Myeloid cells (Mac. and Grans.) show the greatest abundance of KEGG categories.

FIG. 6, comprising FIG. 6A through FIG. 6C, is a series of graphs illustrating chromosomal expression density relative to chromatin state for HSC compared to other cell types. FIG. 6A is a series of chromosomal expression maps and then subtracted against each other. FIG. 6B is a series of chromosomal expression maps, also examined along the X-chromosome. FIG. 6C illustrates differences in expression density between cell types on a per chromosome basis.

FIG. 7, comprising FIG. 7A through FIG. 7C, is a series of charts, illustrating that retroviral transduction of fingerprint transcription factors Ets2 and Zfp105 enforces lineage-specific cell fate. FIG. 7A illustrates the experimental paradigm. FIG. 7B illustrates the overall proportion of transduced cells (transduction, Tx.), and the proportions of transduced myeloid, and lymphoid cells. FIG. 7C illustrates how Zfp105 transplants exhibited a depression in both B- and T-cells, and an overall low level of apparent transduction.

FIG. 8 is a graph depicting the results of retroviral transduction of Chd7 (chromodomain helicase DNA binding protein 7) in HSCs. The percent of peripheral blood cells which undergo engraftment, are CD45.2, B-, T- or myeloid cells is shown.

FIG. 9 is a graph depicting the results of retroviral transduction of Edaradd (ectodysplasin-A receptor)-associated death domain) in HSCs. The percent of peripheral blood cells which undergo engraftment, are CD45.2, B-, T- or myeloid cells is shown.

FIG. 10 is a graph depicting the results of retroviral transduction of Med8 (mediator of RNA polymerase II transcription, subunit 8 homolog) in HSCs. The percent of peripheral blood cells which undergo engraftment, are CD45.2, B-, T- or myeloid cells is shown.

FIG. 11 is a graph depicting the results of retroviral transduction of Med14 (mediator complex subunit 14) in HSCs. The percent of peripheral blood cells which undergo engraftment, are CD45.2, B-, T- or myeloid cells is shown.

FIG. 12 is a graph depicting the results of retroviral transduction of Glis2 (GLIS family zinc finger 2) in HSCs. The percent of peripheral blood cells which undergo engraftment, are CD45.2, B-, T- or myeloid cells is shown.

FIG. 13 is a graph depicting the results of retroviral transduction of Dzip1 (DAZ interacting protein 1) in HSCs. The percent of peripheral blood cells which undergo engraftment, are CD45.2, B-, T- or myeloid cells is shown.

FIG. 14 is a graph depicting the results of retroviral transduction of Tnfaip8l1 (tumor necrosis factor, alpha-induced protein 8-like 1) in HSCs. The percent of peripheral blood cells which undergo engraftment, are CD45.2, B-, T- or myeloid cells is shown.

FIG. 15 is a graph depicting the results of retroviral transduction of 2210016F16Rik in HSCs. The percent of peripheral blood cells which undergo engraftment, are CD45.2, B-, T- or myeloid cells is shown.

FIG. 16 is a graph depicting the results of retroviral transduction of Tbl1x (transducin (beta)-like 1 X-linked) in HSCs. The percent of peripheral blood cells which undergo engraftment, are CD45.2, B-, T- or myeloid cells is shown.

FIG. 17 is a graph depicting the results of retroviral transduction of Mina (myc induced nuclear antigen) in HSCs. The percent of peripheral blood cells which undergo engraftment, are CD45.2, B-, T- or myeloid cells is shown.

### DETAILED DESCRIPTION OF THE INVENTION

The present invention is related to the discovery of biomarkers for myeloid and lymphoid cell lineages. The present invention also discloses biomarkers for differentiated cells, including Natural killer cells, monocytes, B-cells and T-cells. The present invention is further related to the discovery that overexpressing a biomarker in a hematopoietic stem cell (HSC) or a hematopoietic progenitor cell (HPC) directs the differentiation of the cell toward a specific cell lineage. Accordingly, the invention encompasses a method of directing the differentiation of a cell to a specific lineage by introducing a biomarker nucleic acid or a biomarker protein into the cell.

### Definitions:

Unless defined otherwise, all technical and scientific terms used herein have the same meaning as commonly understood by one of ordinary skill in the art to which the invention pertains. Although any methods and materials similar or, equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

As used in this specification and the appended claims, the singular forms “a”, “an”, and “the” include plural referents unless the content clearly dictates otherwise. Thus, for example, reference to “a protein” includes a combination of two or more proteins, and the like.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

A “biomarker” is any gene, protein, or metabolite whose level of expression in a tissue, cell or bodily fluid is unique to that tissue, cell, or bodily fluid.

The phrase “body sample” as used herein, is intended to include any sample comprising a cell, a tissue, or a bodily fluid in which expression of a biomarker can be detected. Examples of such body samples include but are not limited to blood, lymph, biopsies and smears. Samples that are liquid in nature are referred to herein as “bodily fluids.” Body samples may be obtained from a patient by a variety of techniques including, for example, by scraping or swabbing an area or by using a needle to aspirate bodily fluids. Methods for collecting various body samples are well known in the art.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of an mRNA molecule also consists of the nucleotide residues of the mRNA molecule which are matched with an anti-codon region of a transfer RNA molecule during translation of the mRNA molecule or which encode a stop codon. The coding region may thus include nucleotide residues corresponding to amino acid residues which are not present in the mature protein encoded by the mRNA molecule (e.g., amino acid residues in a protein export signal sequence).

“Complementary” as used herein to refer to a nucleic acid, refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The term “DNA” as used herein is defined as deoxyribonucleic acid.

The term “dysregulation” as used herein, describes an abnormality of the cell content of blood. The abnormality may stem from a traumatic event (including hemorrhage) or a disease process (including HIV or cancer). The dysregulation may be detected by a complete blood count (CBC) wherein there is either depletion or over production of a particular blood cell type detected in a blood sample obtained from an individual as compared to a blood sample obtained from one or more normal individuals, or from the same individual at a different time point. Examples of such a dysregulation include elevated red blood cells, low numbers of red blood cells, elevated lymphocytes (leukocytosis) or low numbers of lymphocytes (leucopenia).

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting there from. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “progeny” as used herein refers to a descendent or offspring.

In one use, the term progeny refers to a clonal descendent which is genetically identical to the parent. In another usage, the term progeny refers to cells which have differentiated from the parent cell.

The term “RNA” as used herein is defined as ribonucleic acid.

The term “recombinant DNA” as used herein is defined as DNA produced by joining pieces of DNA from different sources.

The term “recombinant polypeptide” as used herein is defined as a polypeptide produced by using recombinant DNA methods.

By the term “specifically binds,” as used herein, is meant a molecule, such as an antibody, which recognizes and binds to a cell surface molecule or feature, but does not substantially recognize or bind other molecules or features in a sample.

“Stromal cells” as used herein comprise fibroblasts and mesenchymal cells, with or without other cells and elements, and can be seeded prior to, or substantially at the same time as the hematopoietic progenitor cells, therefore establishing conditions that favor the subsequent attachment and growth of hematopoietic progenitor cells. Fibroblasts can be obtained via a biopsy from any tissue or organ, and include fetal fibroblasts. These fibroblasts and mesenchymal cells may be transfected with exogenous DNA that encodes, for example, one of the hematopoietic growth factors described above. Stromal cells can be of lymphoid or non-lymphoid origin.

“Stromal cell conditioned medium” refers to medium in which the aforementioned stromal cells have been incubated. The incubation is performed for a period sufficient to allow the stromal cells to secrete factors into the medium. Such “stromal cell conditioned medium” can then be used to supplement the culture of hematopoietic cells promoting their proliferation and/or differentiation.

As used herein, “conjugated” refers to covalent attachment of one molecule to a second molecule.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

### Description:

The present invention identifies, for the first time, a number of biomarkers which are specific for the myeloid lineage or the lymphoid lineage. The present invention also identifies biomarkers for differentiated cells, including NK cells, monocytes, B-cells, T-cells and monocytes.

The present invention springs from the discovery that hematopoiesis can be directed toward a specific cell lineage by expressing a specific biomarker nucleic acid or protein in a cell. As such, The present invention includes methods of directing the differentiation of a cell toward the myloid or lymphoid cell lineages by expressing these biomarkers in the cell.

### I. Compositions

### Hematopoietic Cells

A cell of the invention can be a pluripotent hematopoietic stem cell (HSC) or a multipotent hematopoietic progenitor cell (HPC). Hematopoietic stem cells and hematopoietic progenitor cells are immature blood cells with the capacity to self-renew and to differentiate into more mature blood cells. Pluripotent HSCs are capable of differentiating into any blood cell type. Multipotent HPCs are capable into differentiating into a blood cell of a particular lineage. HPCs may be either a common myeloid progenitor cell or a common lymphoid progenitor cell. The common myeloid progenitor cell gives rise to cells of the myeloid lineage and the common lymphoid progenitor cell gives rise to cells of the lymphoid lineage. Cells derived from a common myeloid progenitor cell include granulocytes (e.g., neutrophils, eosinophils, basophils), erythrocytes (e.g., reticulocytes, erythrocytes), thrombocytes (e.g., megakaryoblasts, platelet producing megakaryocytes, platelets), and monocytes (e.g. monocytes, macrophages). Cells derived from a common lymphoid progenitor cell include B-cells, T-cells, and natural killer (NK) cells.

A cell of the invention may be derived from bone marrow, peripheral blood (including mobilized peripheral blood), umbilical cord blood, placental blood, fetal liver, embryonic cells (including embryonic stem cells), aortal-gonadal-mesonephros derived cells, and lymphoid soft tissue. Lymphoid soft tissue includes the thymus, spleen, liver, lymph node, skin, tonsil and Peyer's patches.

In certain embodiments, a cell of the invention is autologous (e.g., originate from the same individual). In some embodiments, a cell of the invention is non-autologous. A cell of the present invention may be allogenic, syngenic or xenogenic. “Allogeneic,” as used herein, refers to cells of the same species that differ genetically to the cell in comparison. “Syngeneic,” as used herein, refers to cells of a different subject that are genetically identical to the cell in comparison. “Xenogeneic,” as used herein, refers to cells of a different species to the cell in comparison. In one embodiment of the invention, the cells of the invention are of human origin.

### Biomarkers of Hematopoietic Lineage

In one embodiment of the invention, a biomarker of the invention comprises any gene, nucleic acid, protein, peptide or fragment thereof that is specifically expressed by cells of the myeloid lineage, but not by cells of the lymphoid lineage. In another embodiment of the invention, a biomarker of the invention comprises any gene, nucleic acid, protein, peptide or fragment thereof that is expressed by cells of the lymphoid lineage, but not in cells of the myeloid lineage. Biomarkers of particular interest include genes and proteins involved in hematopoiesis. In some embodiments, the biomarker is an enzyme, a protein associated with cellular differentiation, or a transcription factor. In another embodiment of the invention, expression of a biomarker in a cell of the invention directs the differentiation of that cell to a specific cell lineage.

“Biomarker nucleic acids,” as used herein, include DNA comprising the entire or partial sequence of the nucleic acid sequence encoding a biomarker, or the complement of such a sequence. Biomarker nucleic acids useful in the invention should be considered to include both DNA and RNA comprising the entire or partial sequence of any of the nucleic acid sequences of interest.

“Biomarker proteins”, as used herein, should be considered to comprise the entire or partial amino acid sequence of any of the biomarker proteins or polypeptides.

In one embodiment of the invention, biomarker nucleic acids include, but are not limited to, nucleic acids encoding Ets2, Zfp105, Chd7 (chromodomain helicase DNA binding protein 7), Edaradd (EDAR (ectodysplasin-A receptor)-associated death domain), Med8 (mediator of RNA polymerase II transcription, subunit 8 homolog), Med14 (mediator complex subunit 14), Glis2 (GLIS family zinc finger 2), Dzip1 (DAZ interacting protein 1), Tnfaip8l1 (tumor necrosis factor, alpha-induced protein 8-like 1), 2210016F16Rik , Tbl1x (transducin (beta)-like 1 X-linked), and Mina (myc induced nuclear antigen).

Ets2 is a member of the ETS family of transcription factors, many of which have been implicated in tumor progression. Ets2 is implicated in acute myeloid leukemia and has been shown to play a role in inhibiting apoptosis of macrophages (Baldus et al., 2004, Proc. Natl. Acad. Sci. U.S.A. 101:3915-1920; Sevilla et al., 1999, Mol. Cell. Biol. 19:2624-2634).

Zfp105 has previously been shown to be involved in spermatogenesis (Przybborski et al., 1998, Mamm. Genome 9:758-62).

Chd7 (chromodomain helicase DNA binding protein 7), a B-cell fingerprint gene, is a putative transcription factor associated with CHARGE syndrome in humans. CHARGE is a complex human disorder with such clinical features as coloboma of the eye, choanal atresia or stenosis, abnormalities of the cranial nerves, otological deformities, and defects of one or more major organ systems. At least one gene has been identified with CHARGE. Heterozygous Chd7 knock-out mice display the symptoms of CHARGE syndrome.

Edaradd (EDAR (ectodysplasin-A receptor)-associated death domain), a B-cell fingerprint gene, is a cytoplasmic receptor previously implicated in fertility and hair follicle development in mice.

Med8 (mediator of RNA polymerase II transcription, subunit 8 homolog), a Differentiated fingerprint gene, skews differentiation toward the myeloid lineages. Myeloid skewing is consistent with a role in stem cell expansion. Med8 is a putative transcription factor that may be part of the mediator complex.

Med14 (mediator complex subunit 14), an HSC fingerprint gene, is a putative transcription factor that may be part of the mediator complex.

Glis2 (GLIS family zinc finger 2), an HSC fingerprint gene, is a transcription factor known to negatively regulate transcription. Homozygous Glis2 knock-outs exhibit kidney atrophy and cysts.

Dzip1 (DAZ interacting protein 1), a naïve T-cell fingerprint gene, has putative nucleic acid binding capacity and therefore is classified as a potential transcription factor.

Tnfaip8l1 (tumor necrosis factor, alpha-induced protein 8-like 1) is a naïve T-cell fingerprint gene.

2210016F16Rik, an activated T-cell fingerprint gene, has no known function and belongs to a group of genes (Riken genes) identified by large scale sequencing of messenger RNA.

Tbl1x (transducin (beta)-like 1 X-linked), an activated T-cell fingerprint gene, is a transcription factor involved in nuclear receptor-mediated transcription.

Mina (myc induced nuclear antigen), an activated T-cell fingerprint gene, is localized to the nucleus and may play a role in cellular proliferation.

In another embodiment of the invention, a biomarker comprises a biomarker protein, including, but not limited to, Ets2, Zfp105, Chd7, Edaradd, Med8, Med14, Glis2, Dzip1, Tnfaip8l1, 2210016F16Rik, Tbl1x, and Mina.

The present invention also provides for analogs of polypeptides which comprise a biomarker protein. Analogs may differ from naturally occurring proteins or polypeptides by conservative amino acid sequence differences or by modifications which do not affect sequence, or by both. For example, conservative amino acid changes may be made, which although they alter the primary sequence of the protein or polypeptide, do not normally alter its function (e.g., secretion and capable of blocking virus infection). Conservative amino acid substitutions typically include substitutions within the following groups:
glycine, alanine;
valine, isoleucine, leucine;
aspartic acid, glutamic acid;
asparagine, glutamine;
serine, threonine;
lysine, arginine;
phenylalanine, tyrosine.

Modifications (which do not normally alter primary sequence) include in vivo, or in vitro, chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

The present invention should also be construed to encompass “mutants,” “derivatives,” and “variants” of the biomarker proteins of the invention (or of the DNA encoding the same) which mutants, derivatives and variants are altered in one or more amino acids (or, when referring to the nucleotide sequence encoding the same, are altered in one or more base pairs) such that the resulting peptide (or DNA) is not identical to the sequences recited herein, but has the same biological property as the biomarker proteins disclosed herein, in that the proteins have biological/biochemical properties. A biological property of the polypeptides of the present invention should be construed but not be limited to include, the ability to mediate normal hematopoiesis.

Further, the invention should be construed to include naturally occurring variants or recombinantly derived mutants of biomarker proteins sequences, which variants or mutants render the polypeptide encoded thereby either more, less, or just as biologically active as wild type biomarker proteins.

The present invention should not be construed to be limited to the biomarkers nucleic acids and biomarker proteins recited herein, but rather should be construed to encompass any biomarker, which, when expressed in a cell of the invention directs the differentiation of said cell toward a selected cell lineage. Methods for identifying such biomarkers are within the skill of the art, and are described herein.

### Biomarker Nucleic Acids: Synthesis

Any number of procedures may be used for the generation of an isolated biomarker nucleic acid as well as derivative or variant forms of an isolated biomarker nucleic acid, using recombinant DNA methodology well known in the art (see Sambrook et al., 2001, Molecular Cloning: A Laboratory Manual, Cold Spring Harbor Laboratory Press, New York; Ausubel et al., 2001, Current Protocols in Molecular Biology, Green & Wiley, New York) and by direct synthesis. For recombinant and in vitro transcription, DNA encoding RNA molecules can be obtained from known clones, by synthesizing a DNA molecule encoding an RNA molecule, or by cloning the gene encoding the RNA molecule. Techniques for in vitro transcription of RNA molecules and methods for cloning genes encoding known RNA molecules are described by, for example, Sambrook et al.

An isolated biomarker nucleic acid of the present invention can be produced using conventional nucleic acid synthesis or by recombinant nucleic acid methods known in the art and described elsewhere herein (2001, Molecular Cloning: A Laboratory Manual, Cold Spring Harbor Laboratory Press, New York) and Ausubel et al. (2001, Current Protocols in Molecular Biology, Green & Wiley, New York).

As an example, a method for synthesizing nucleic acids de novo involves the organic synthesis of a nucleic acid from nucleoside derivatives. This synthesis may be performed in solution or on a solid support. One type of organic synthesis is the phosphotriester method, which has been used to prepare gene fragments or short genes. In the phosphotriester method, oligonucleotides are prepared which can then be joined together to form longer nucleic acids. For a description of this method, see Narang, et al., (1979, Meth. Enzymol., 68: 90) and U.S. Pat. No. 4,356,270. The phosphotriester method can be used in the present invention to synthesize an isolated snRNA.

In addition, the compositions of the present invention can be synthesized in whole or in part, or an isolated biomarker nucleic acid can be conjugated to another nucleic acid using organic synthesis such as the phosphodiester method, which has been used to prepare a tRNA gene. See Brown, et al. (1979, Meth. Enzymol., 68: 109) for a description of this method. As in the phosphotriester method, the phosphodiester method involves synthesis of oligonucleotides which are subsequently joined together to form the desired nucleic acid.

A third method for synthesizing nucleic acids, described in U.S. Pat. No. 4,293,652, is a hybrid of the above-described organic synthesis and molecular cloning methods. In this process, the appropriate number of oligonucleotides to make up the desired nucleic acid sequence is organically synthesized and inserted sequentially into a vector which is amplified by growth prior to each succeeding insertion.

In addition, molecular biological methods, such as using a nucleic acid as a template for a PCR or LCR reaction, or cloning a nucleic acid into a vector and transforming a cell with the vector can be used to make large amounts of the nucleic acid of the present invention.

Any of a variety of procedures may be used to molecularly clone biomarker cDNA. These methods include, but are not limited to, direct functional expression of a biomarker gene, including but not limited to Ets2, Zfp105, Chd7, EDAR, Med8, Med14, Glis2, Dzip1, Tnfaip8l1, 2210016F16Rik, Tbl1x, and Mina, following the construction of a biomarker-containing cDNA library in an appropriate expression vector system.

It is readily apparent to those skilled in the art that suitable cDNA libraries may be prepared from cells or cell lines which express a biomarker of interest. By way of a non-limiting example, cells or cell-lines which express Ets2 would be useful in the preparation of a cDNA library. Similarly, cells or cell lines which express Zfp105 would be useful in the preparation of a cDNA library.

Preparation of cDNA libraries can be performed by standard techniques well known in the art. Well known cDNA library construction techniques can be found for example, in Maniatis, T., Fritsch, E. F., Sambrook, J., Molecular Cloning: A Laboratory Manual (Cold Spring Harbor Laboratory, Cold Spring Harbor, N.Y., 1982).

It is also readily apparent to those skilled in the art that DNA encoding a biomarker may also be isolated from a suitable genomic DNA library. Construction of genomic DNA libraries can be performed by standard techniques well known in the art. Well known genomic DNA library construction techniques can be found in Maniatis, T., Fritsch, E. F., Sambrook, J. in Molecular Cloning: A Laboratory Manuel (Cold Spring Harbor Laboratory, Cold Spring Harbor, N.Y., 1982).

Biomarker molecules may also be obtained by recombinantly engineering them from DNA encoding the partial or complete amino acid sequence of a biomarker. Using recombinant DNA techniques, DNA molecules are constructed which encode at least a portion of a biomarker molecule such at Ets2, Zfp105, Chd7, EDAR, Med8, Med14, Glis2, Dzip1, Tnfaip8l1, 2210016F16Rik, Tbl1x, and Mina. Standard recombinant DNA techniques are used such as those found in Maniatis, et al., supra.

The cloned biomarker cDNA obtained through the methods described above may be recombinantly expressed by molecular cloning into an expression vector containing a suitable promoter and other appropriate transcription regulatory elements, and transferred into prokaryotic or eukaryotic host cells to produce recombinant biomarker. Techniques for such manipulations are fully described in Maniatis, T, et al., supra, and are well known in the art.

In other related aspects, the invention includes an isolated nucleic acid encoding a biomarker, operably linked to a nucleic acid comprising a promoter/regulatory sequence such that the nucleic acid is preferably capable of directing expression of the biomarker protein encoded by the nucleic acid. Thus, the invention encompasses expression vectors and methods for the introduction of exogenous DNA into cells with concomitant expression of the exogenous DNA in the cells such as those described, for example, in Sambrook et al. (2001, Molecular Cloning: A Laboratory Manual, Cold Spring Harbor Laboratory, New York), and in Ausubel et al. (1997, Current Protocols in Molecular Biology, John Wiley & Sons, New York). The incorporation of a desired polynucleotide into a vector and the choice of vectors is well-known in the art as described in, for example, Sambrook et al., supra, and Ausubel et al., supra.

The biomarker polynucleotide can be cloned into a number of types of vectors. However, the present invention should not be construed to be limited to any particular vector. Instead, the present invention should be construed to encompass a wide plethora of vectors which are readily available and/or well-known in the art. For example, a biomarker polynucleotide of the invention can be cloned into a vector including, but not limited to a plasmid, a phagemid, a phage derivative, an animal viruse, and a cosmid. Vectors of particular interest include expression vectors, replication vectors, probe generation vectors, and sequencing vectors.

In specific embodiments, the expression vector is selected from the group consisting of a viral vector, a bacterial vector and a mammalian cell vector. Numerous expression vector systems exist that comprise at least a part or all of the compositions discussed above. Prokaryote- and/or eukaryote-vector based systems can be employed for use with the present invention to produce polynucleotides, or their cognate polypeptides. Many such systems are commercially and widely available.

Further, the expression vector may be provided to a cell in the form of a viral vector. Viral vector technology is well known in the art and is described, for example, in Sambrook et al. (2001), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

Additional promoter elements, i.e., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either co-operatively or independently to activate transcription.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. No. 4,683,202, U.S. Pat. No. 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2001). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

A promoter sequence exemplified in the experimental examples presented herein is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, Moloney virus promoter, the avian leukemia virus promoter, Epstein-Barr virus immediate early promoter, Rous sarcoma virus promoter, as well as human gene promoters such as, but not limited to, the actin promoter, the myosin promoter, the hemoglobin promoter, and the muscle creatine promoter. Further, the invention should not be limited to the use of constitutive promoters. Inducible promoters are also contemplated as part of the invention. The use of an inducible promoter in the invention provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. Further, the invention includes the use of a tissue specific promoter, which promoter is active only in a desired tissue. Tissue specific promoters are well known in the art and include, but are not limited to, the HER-2 promoter and the PSA associated promoter sequences.

In order to assess the expression of the biomarker, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000 FEBS Lett. 479:79-82). Suitable expression systems are well known and may be prepared using well known techniques or obtained commercially. Internal deletion constructs may be generated using unique internal restriction sites or by partial digestion of non-unique restriction sites. Constructs may then be transfected into cells that display high levels of siRNA polynucleotide and/or polypeptide expression. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

### Biomarker Proteins: Synthesis

Biomarker proteins useful in the invention, including, but not limited to, Ets2, Zfp105, Chd7, EDAR, Med8, Med14, Glis2, Dzip1, Tnfaip8l1, 2210016F16Rik, Tbl1x, and Mina, may be obtained using standard methods known to the skilled artisan. Such methods include chemical organic synthesis or biological means. Biological means include purification from a biological source, recombinant synthesis and in vitro translation systems, using methods well known in the art.

A peptide may be chemically synthesized by Merrifield-type solid phase peptide synthesis. This method may be routinely performed to yield peptides up to about 60-70 residues in length, and may, in some cases, be utilized to make peptides up to about 100 amino acids long. Larger peptides may also be generated synthetically via fragment condensation or native chemical ligation (Dawson et al., 2000, Ann. Rev. Biochem. 69:923-960). An advantage to the utilization of a synthetic peptide route is the ability to produce large amounts of peptides, even those that rarely occur naturally, with relatively high purities, i.e., purities sufficient for research, diagnostic or therapeutic purposes.

Solid phase peptide synthesis is described by Stewart et al. in Solid Phase Peptide Synthesis, 2nd Edition, 1984, Pierce Chemical Company, Rockford, Ill.; and Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and coupling thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group, such as formation into a carbodiimide, a symmetric acid anhydride, or an “active ester” group, such as hydroxybenzotriazole or pentafluorophenyl esters.

Examples of solid phase peptide synthesis methods include the BOC method, which utilizes tert-butyloxcarbonyl as the α-amino protecting group, and the FMOC method, which utilizes 9-fluorenylmethyloxcarbonyl to protect the α-amino of the amino acid residues. Both methods are well-known by those of skill in the art.

Incorporation of N- and/or C-blocking groups may also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin, so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB (divinylbenzene), resin, which upon hydrofluoric acid (HF) treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being affected by trifluoroacetic acid (TFA) in dicholoromethane. Esterification of the suitably activated carboxyl function, e.g. with dicyclohexylcarbodiimide (DCC), can then proceed by addition of the desired alcohol, followed by de-protection and isolation of the esterified peptide product.

Incorporation of N-terminal blocking groups may be achieved while the synthesized peptide is still attached to the resin, for instance by treatment with a suitable anhydride and nitrile. To incorporate an acetyl blocking group at the N-terminus, for instance, the resin-coupled peptide can be treated with 20% acetic anhydride in acetonitrile. The N-blocked peptide product may then be cleaved from the resin, de-protected and subsequently isolated.

Prior to its use in accordance with the invention, a biomarker protein, peptide, or fragment thereof is purified to remove contaminants. Any one of a number of a conventional purification procedures may be used to attain the required level of purity including, for example, reversed-phase high-pressure liquid chromatoraphy (HPLC) using an alkylated silica column such as C₄-, C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate polypeptides based on their charge. Affinity chromatography is also useful in purification procedures.

Proteins and peptides may be modified using ordinary molecular biological techniques to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The polypeptides useful in the invention may further be conjugated to non-amino acid moieties that are useful in their application. In particular, moieties that improve the stability, biological half-life, water solubility, and immunologic characteristics of the peptide are useful. A non-limiting example of such a moiety is polyethylene glycol (PEG).

### II. Methods

The present invention provides methods for directing the differentiation of a cell of the invention to a specific cell lineage by overexpressing a biomarker nucleic acid in the cell. The present invention also provides methods for directing the differentiation of a cell of the invention to a specific cell lineage by overexpressing a biomarker protein in the cell.

In one embodiment, the differentiation of a cell of the invention is directed to the myeloid lineage. In another embodiment of the invention, the differentiation of a cell of the invention is directed to the lymphoid lineage.

One embodiment of the invention comprises directing the differentiation of a cell of the invention into a natural kill (NK) cell by overexpressing a nucleic acid encoding Zinc finger protein (Zfp) 105 the cell. Another embodiment of the invention comprises directing the differentiation of a cell of the invention into a NK cell by overexpressing Zfp105 protein in the cell.

Another embodiment of the invention comprises directing the differentiation of a cell of the invention into a monocyte cell by overexpressing a nucleic acid encoding Ets2 in the cell. Another embodiment of the invention comprises directing the differentiation of a cell of the invention into a monocyte cell by overexpressing Ets2 protein in the cell.

Yet another embodiment of the invention comprises directing the differentiation of a cell of the invention into a B-cell by overexpressing a nucleic acid encoding Chd7 in the cell. Another embodiment of the invention comprises directing the differentiation of a cell of the invention into a B-cell by overexpressing Chd7 protein in the cell.

One embodiment of the invention comprises directing the differentiation of a cell of the invention into a B-cell by overexpressing a nucleic acid encoding EDAR in the cell. Another embodiment of the invention comprises directing the differentiation of a cell of the invention into a B-cell by overexpressing EDAR protein in the cell.

Still another embodiment of the invention comprises directing the differentiation of a cell of the invention to a myeloid lineage by overexpressing a nucleic acid encoding Med8 in the cell. Another embodiment of the invention comprises directing the differentiation of a cell of the invention to a myeloid lineage by overexpressing Med8 protein in the cell.

One embodiment of the invention comprises directing the differentiation of a cell of the invention to a myeloid lineage by overexpressing a nucleic acid encoding Med14 in the cell. Another embodiment of the invention comprises directing the differentiation of a cell of the invention to a myeloid lineage by overexpressing Med14 protein in the cell.

Another embodiment of the invention comprises directing the differentiation of a cell of the invention to a myeloid lineage by overexpressing a nucleic acid encoding Glis2 in the cell. Another embodiment of the invention comprises directing the differentiation of a cell of the invention to a myeloid lineage by overexpressing Glis2 protein in the cell.

One embodiment of the invention comprises directing the differentiation of a cell of the invention into a B-cell by overexpressing a nucleic acid encoding Dzip1 in the cell. Another embodiment of the invention comprises directing the differentiation of a cell of the invention into a B-cell by overexpressing Dzip1 protein in the cell.

Another embodiment of the invention comprises directing the differentiation of a cell of the invention to a myeloid lineage by overexpressing a nucleic acid encoding Tnfaip8l1 in the cell. Another embodiment of the invention comprises directing the differentiation of a cell of the invention to a myeloid lineage by overexpressing Tnfaip8l1 protein in the cell.

One embodiment of the invention comprises directing the differentiation of a cell of the invention into a B-cell by overexpressing a nucleic acid encoding 2210016F16Rik in the cell. Another embodiment of the invention comprises directing the differentiation of a cell of the invention into a B-cell by overexpressing 2210016F16Rik protein in the cell.

One embodiment of the invention comprises directing the differentiation of a cell of the invention into a B-cell by overexpressing a nucleic acid encoding Tbl1x (transducin (beta)-like 1 X-linked) in the cell. Another embodiment of the invention comprises directing the differentiation of a cell of the invention into a B-cell by overexpressing Tbl1x protein in the cell.

Another embodiment of the invention comprises directing the differentiation of a cell of the invention to a myeloid lineage by overexpressing nucleic acid encoding Mina (myc induced nuclear antigen) in the cell. Another embodiment of the invention comprises directing the differentiation of a cell of the invention to a myeloid lineage by overexpressing Mina protein in the cell.

In one embodiment of the invention, a biomarker nucleic acid is administered to a mammal whereby the biomarker nucleic acid enters a cell of the invention, is expressed therein, and thereby directs the cell's differentiation. In another embodiment of the present invention, a biomarker protein is administered to a mammal whereby the biomarker protein contacts or is introduced to a cell of the invention, thereby directing the cell's differentiation. In one embodiment the mammal is a human.

In another embodiment of the present invention, a cell of the invention is harvested from a mammal and brought into contact with a biomarker nucleic acid whereby the biomarker nucleic acid directs the cell's differentiation to a cell lineage. In another embodiment of the present invention, a cell of the invention is harvested from a mammal and brought into contact with a biomarker protein whereby the biomarker protein directs the cell's differentiation to a cell lineage. A cell of the invention may be cultured, expanded and allowed to proliferate and differentiate ex vivo prior to being returned to the mammal. In one embodiment, the mammal is a human.

The culture of a cell of the invention preferably occurs under conditions that increase the number of such cells and/or the colony forming potential of such cells. The conditions used refer to a combination of conditions known in the art (e.g., temperature, CO₂ and O₂ content, nutritive media, etc.). The time sufficient to increase the number of cells is a time that can be easily determined by a person skilled in the art, and can vary depending upon the original number of cells seeded. As an example, discoloration of the media can be used as an indicator of confluency. Additionally, different volumes of the blood product can be cultured under identical conditions, and cells can be harvested and counted over regular time intervals, thus generating the “control curves.” These “control curves” can be used to estimate cell numbers in subsequent occasions.

The conditions for determining colony forming potential are similarly determined. Colony forming potential is the ability of a cell to form progeny. Assays for this are well known to those of ordinary skill in the art and include seeding cells into a semi-solid substrate or media, treating them with growth factors and counting the number of colonies.

In all of the culturing methods according to the invention, except as otherwise provided, the liquid cell culture media utilized herein are conventional media for culturing cells. Examples include, but are not limited to, RPMI, DMEM, ISCOVES, etc. Typically these media are supplemented with human or animal plasma or serum. Such plasma or serum can contain small amounts of growth factors. The media used according to the present invention, however, can depart, in certain embodiments, from that used conventionally in the prior art. In particular, a cell of the invention can be cultured on matrices and devices for extended periods of time without the need for adding any exogenous growth agents (other than those which may be contained in plasma or serum, hereinafter “serum”), without inoculating the environment of the culture with stromal cells and without using stromal cell conditioned media. In a continuous cell culture system, it is preferred to add and remove the cell culture media from the vessel at a rate of less than ½ volume change per day.

The growth agents of particular interest in connection with the present invention are hematopoietic growth factors. By hematopoietic growth factors, it is meant factors that influence the survival, proliferation or differentiation of hematopoietic cells. Growth agents that affect only survival and proliferation, but are not believed to promote differentiation, include but are not limited to the interleukins 3, 6 and 11, stem cell ligand and FLT-3 ligand. Hematopoietic growth factors that promote differentiation include but are not limited to the colony stimulating factors such as GMCSF, GCSF, MCSF, Tpo, Epo, Oncostatin M, and interleukins other than IL-3, 6 and 11. The foregoing factors are well known to those of ordinary skill in the art. Most are commercially available. They can be obtained by purification, by recombinant methodologies or can be derived or synthesized synthetically.

It is possible according to the invention to preserve a cell of the invention and to stimulate the expansion and differentiation of a cell of the invention using the compositions and methods of the present invention. Once a cell of the invention has been contacted with a biomarker nucleic acid or a biomarker protein to direct differentiation of the cell to a particular cell lineage, the cell can be expanded and, for example, be specifically identified and/or selected by the biomarkers described herein before being returned to the body to supplement, replenish, or restore a patient's myeloid or lymphoid cell populations. This might be appropriate, for example, after an individual has undergone chemotherapy.

In another embodiment of the invention, a cell of the invention may be cultured for an extended period of time, and aliquots of the cultured cells are harvested spaced apart in time or intermittently. Harvesting cells encompasses dislodging or separation of cells from the matrix. This can be accomplished using a number of methods, such as enzymatic, centrifugal, electrical or by size, or by flushing of the cells using the media in which the cells are incubated. The cells can be further collected and separated. “Harvesting steps spaced apart in time” or “intermittent harvest of cells” is meant to indicate that a portion of the cells are harvested, leaving behind another portion of cells for their continuous culture in the established media, maintaining a continuous source of the original cells and their characteristics. Harvesting “at least a portion of” means harvesting a subpopulation of or the entirety of. Thus, as will be understood by one of ordinary skill in the art, the invention can be used to expand the number of cells, all the while harvesting portions of those cells being expanded for treatment to develop even larger populations of differentiated cells.

The present invention contemplates the use of the compositions and methods of the invention in the treatment of a subject, preferably a mammal, more preferably a human, suffering from a disease or disorder wherein the directed differentiation of a cell of the invention would be beneficial the subject. The term “beneficial” as used herein, refers to a reduction in the severity or frequency of symptoms, a restoration of health, or a therapeutic improvement. Some non-limiting examples where the compositions and methods of the present invention are useful include treating any disease or disorder where the blood cell count of a subject is dysregulated, includin, but not limited to diseases and disorders where blood cells are depleted, such as anemia, destroyed by a pathogen, such as Aquired Immunodeficiency Syndrome (AIDS), or where hematopoiesis is dysregulated, such as in cancer (e.g. leukemias and lymphomas). Also contemplated is the use of the compositions and methods of the present invention in situations where a subject's blood cell count is dysregulated due to a therapeutic treatment or regimine, such as chemotherapy or radiation therapy.

### Pharmaceutical Compositions and Methods of Use

Compositions comprising biomarker nucleic acids or biomarker proteins can be incorporated into pharmaceutical compositions suitable for administration. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. Supplementary active compounds can also be incorporated into the compositions.

A formulated composition comprising a biomarker nucleic acid or biomarker protein can assume a variety of states. In some examples, the composition is at least partially crystalline, uniformly crystalline, and/or anhydrous (e.g., less than 80, 50, 30, 20, or 10% water). In another example, the biomarker nucleic acid or biomarker protein is in an aqueous phase, e.g., in a solution that includes water, this form being the preferred form for administration via inhalation.

The aqueous phase or the crystalline compositions can be incorporated into a delivery vehicle, e.g., a liposome (particularly for the aqueous phase), or a particle (e.g., a microparticle as can be appropriate for a crystalline composition). Generally, the composition comprising a biomarker nucleic acid or biomarker protein is formulated in a manner that is compatible with the intended method of administration.

A biomarker nucleic acid or biomarker protein preparation can be formulated in combination with another agent, e.g., another therapeutic agent or an agent that stabilizes a biomarker nucleic acid or biomarker protein agent, e.g., a protein that complexes with the biomarker nucleic acid or biomarker protein agent. Still other agents include chelators, e.g., EDTA (e.g., to remove divalent cations such as Mg²⁺), salts, RNAse inhibitors (e.g., a broad specificity RNAse inhibitor such as RNAsin) and so forth.

In one embodiment, the biomarker nucleic acid or biomarker protein agent is administered in conjunction with another therapeutic agent such as an antibiotic, an antiviral, an anti-inflammatory, a chemotherapeutic agent, a pain relieving agent, or any other therapeutic compound useful in the treatment of a particular disease or disorder. The biomarker nucleic acid or biomarker protein agent may be administered as part of an on-going treatment regimen or therapy for a particular disease such as chemotherapy or radiation therapy for various cancers.

Pharmaceutical compositions of the invention include a pharmaceutical carrier that may contain a variety of components that provide a variety of functions, including regulation of drug concentration, regulation of solubility, chemical stabilization, regulation of viscosity, absorption enhancement, regulation of pH, and the like. The pharmaceutical carrier may comprise a suitable liquid vehicle or excipient and an optional auxiliary additive or additives. The liquid vehicles and excipients are conventional and commercially available. Illustrative thereof are distilled water, physiological saline, aqueous solutions of dextrose, and the like. For water soluble formulations, the pharmaceutical composition preferably includes a buffer such as a phosphate buffer, or other organic acid salt, preferably at a pH of between about 7 and 8. For formulations containing weakly soluble antisense compounds, micro-emulsions may be employed, for example by using a nonionic surfactant such as polysorbate 80 in an amount of 0.04-0.05% (w/v), to increase solubility. Other components may include antioxidants, such as ascorbic acid, hydrophilic polymers, such as, monosaccharides, disaccharides, and other carbohydrates including cellulose or its derivatives, dextrins, chelating agents, such as EDTA, and like components well known to those in the pharmaceutical sciences, e.g., Remington's Pharmaceutical Science, latest edition (Mack Publishing Company, Easton, Pa.).

Nucleic acid biomarkers and protein biomarkers contemplated in the invention include the pharmaceutically acceptable salts thereof, including those of alkaline earths, e.g., sodium or magnesium, ammonium or NM₄⁺, wherein X is C₁-C₄ alkyl. Other pharmaceutically acceptable salts include organic carboxylic acids such as acetic, lactic, tartaric, malic, isethionic, lactobionic, and succinic acids; organic sulfonic acids such as methanesulfonic, ethanesulfonic, and benzenesulfonic; and inorganic acids such as hydrochloric, sulfuric, phosphoric, and sulfamic acids. Pharmaceutically acceptable salts of a compound having a hydroxyl group include the anion of such compound in combination with a suitable cation such as Na⁺, NH₄⁺, or the like.

The biomarker nucleic acid or biomarker protein agent of the invention may be administered into a recipient in a wide variety of ways. Preferred modes of administration are parenteral, intraperitoneal, intravenous, intradermal, epidural, intraspinal, intrastemal, intra-articular, intra-synovial, intrathecal, intra-arterial, intracardiac, intramuscular, intranasal, subcutaneous, intraorbital, intracapsular, topical, transdermal patch, via rectal, vaginal or urethral administration including via suppository, percutaneous, nasal spray, surgical implant, internal surgical paint, infusion pump, or via catheter.

Suitable methods for nucleic acid delivery according to the present invention are believed to include virtually any method by which a nucleic acid (e.g., DNA, RNA, including viral and nonviral vectors) can be introduced into an organelle, a cell, a tissue or an organism, as described herein or as would be known to one of ordinary skill in the art. For example, the biomarker nucleic acid can be administered to the subject either as a naked oligonucleotide agent, in conjunction with a delivery reagent, or as a recombinant plasmid or viral vector which expresses the oligonucleotide agent.

In addition to administration with conventional carriers, the biomarker nucleic acid may be administered by a variety of specialized delivery techniques.

Sustained release systems suitable for use with the pharmaceutical compositions of the invention include semi-permeable polymer matrices in the form of films, microcapsules, or the like, comprising polylactides, copolymers of L-glutamic acid and gamma-ethyl-L-glutamate, poly(2-hydroxyethyl methacrylate), and like materials, e.g., Rosenberg et al., International application PCT/US92/05305.

The biomarker nucleic acid or biomarker protein agent may be encapsulated in liposomes for therapeutic delivery, as described for example in Liposome Technology, Vol. II, Incorporation of Drugs, Proteins, and Genetic Material, CRC Press. The biomarker nucleic acid or biomarker protein agent, depending upon its solubility, may be present both in the aqueous layer and in the lipidic layer, or in what is generally termed a liposomic suspension. The hydrophobic layer, generally but not exclusively, comprises phospholipids such as lecithin and sphingomyelin, steroids such as cholesterol, ionic surfactants such as diacetylphosphate, stearylamine, or phosphatidic acid, and/or other materials of a hydrophobic nature.

The biomarker nucleic acid or biomarker protein agent may be conjugated to poly(L-lysine) to increase cell penetration. Such conjugates are described by Lemaitre et al., Proc. Natl. Acad. Sci. USA, 84, 648-652 (1987). The procedure requires that the 3′-terminal nucleotide be a ribonucleotide. The resulting aldehyde groups are then randomly coupled to the epsilon-amino groups of lysine residues of poly(L-lysine) by Schiff base formation, and then reduced with sodium cyanoborohydride. This procedure converts the 3′-terminal ribose ring into a morpholine structure antisense oligomers.

A biomarker nucleic acid or biomarker protein agent of the invention also includes conjugates with appropriate ligand-binding molecules. The biomarker nucleic acid or biomarker protein agent may be conjugated for therapeutic administration to ligand-binding molecules which recognize cell-surface molecules, such as according to International Patent Application WO 91/04753. The ligand-binding molecule may comprise, for example, an antibody against a cell surface antigen, an antibody against a cell surface receptor, a growth factor having a corresponding cell surface receptor, an antibody to such a growth factor, or an antibody which recognizes a complex of a growth factor and its receptor. Methods for conjugating ligand-binding molecules to oligonucleotides are detailed in WO 91/04753.

An pharmaceutical composition comprising a biomarker nucleic acid or biomarker protein of the invention can be administered at a unit dose less than about 75 mg per kg of bodyweight, or less than about 70, 60, 50, 40, 30, 20, 10, 5, 2, 1, 0.5, 0.1, 0.05, 0.01, 0.005, 0.001, or 0.0005 mg per kg of bodyweight, and less than 200 nmol of a biomarker nucleic acid or biomarker protein per kg of bodyweight, or less than 1500, 750, 300, 150, 75, 15, 7.5, 1.5, 0.75, 0.15, 0.075, 0.015, 0.0075, 0.0015, 0.00075, 0.00015 nmol of a biomarker nucleic acid or biomarker protein per kg of bodyweight. It is understood that any and all whole or partial integers between the ranges set forth here are included herein. The unit dose, for example, can be administered by injection (e.g., intravenous or intramuscular, intrathecally, or directly into an organ), inhalation, or a topical application.

Delivery of an a biomarker nucleic acid or biomarker protein directly to an organ can be at a dosage on the order of about 0.00001 mg to about 3 mg per organ, or preferably about 0.0001-0.001 mg per organ, about 0.03-3.0 mg per organ, about 0.1-3.0 mg per organ or about 0.3-3.0 mg per organ.

In one embodiment, the unit dose is administered less frequently than once a day, e.g., less than every 2, 4, 8 or 30 days. In another embodiment, the unit dose is not administered with a frequency (e.g., not a regular frequency). For example, the unit dose may be administered a single time. Alternatively, it is possible to administer the composition with a frequency of less than once per day, or, for some instances, only once for the entire therapeutic regimen.

In one embodiment, a subject is administered an initial dose, and one or more maintenance doses of a biomarker nucleic acid or biomarker protein. The maintenance dose or doses are generally lower than the initial dose, e.g., one-half less of the initial dose. A maintenance regimen can include treating the subject with a dose or doses ranging from 0.01 μg to 75 mg/kg of body weight per day, e.g., 70, 60, 50, 40, 30, 20, 10, 5, 2, 1, 0.5, 0.1, 0.05, 0.01, 0.005, 0.001, or 0.0005 mg per kg of bodyweight per day. The maintenance doses are preferably administered no more than once every 5, 10, or 30 days. Further, the treatment regimen may last for a period of time which will vary depending upon the nature of the particular disease, its severity and the overall condition of the patient. In preferred embodiments the dosage may be delivered no more than once per day, e.g., no more than once per 24, 36, 48, or more hours, e.g., no more than once every 5 or 8 days. Following treatment, the patient can be monitored for changes in his condition and for alleviation of the symptoms of the disease state. The dosage of the compound may either be increased in the event the patient does not respond significantly to current dosage levels, or the dose may be decreased if an alleviation of the symptoms of the disease state is observed, if the disease state has been ablated, or if undesired side-effects are observed.

The effective dose can be administered in a single dose or in two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracistemal or intracapsular), or reservoir may be advisable.

Certain factors may influence the dosage required to effectively treat a subject, including but not limited to the severity of the disease or disorder, previous treatments, the general health and/or age of the subject, and other diseases present. It will also be appreciated that the effective dosage of a biomarker nucleic acid or biomarker protein used for treatment may increase or decrease over the course of a particular treatment. Changes in dosage may result and become apparent from the results of diagnostic assays. For example, the subject can be monitored after administering a biomarker nucleic acid or biomarker protein composition. Based on information from the monitoring, an additional amount of biomarker nucleic acid or biomarker protein composition can be administered.

Dosing is dependent on severity and responsiveness of the disease condition to be treated, with the course of treatment lasting from several days to several months, or until a cure is effected or a diminution of disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual compounds, and can generally be estimated based on EC₅₀s found to be effective in in vitro and in vivo animal models.

A pharmaceutical composition of the invention may further comprise one or more additional pharmaceutically active agents. The compositions and methods of the present invention can be used in combination with other treatment regimens, including virostatic and virotoxic agents, antibiotic agents, antifungal agents, anti-inflammatory agents, as well as combination therapies, and the like. The invention can also be used in combination with other treatment modalities, such as chemotherapy, cryotherapy, hyperthermia, radiation therapy, and the like.

### Experimental Examples

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The materials and methods employed in the experiments disclosed herein are now described.

### Mice and Cell Purification

All cell types were purified according to published protocols using a combination of magnetic and flow cytometric cell sorting to achieve at least 95% purity. C57BV6-CD45.1 mice were housed in a specific pathogen free barrier and fed autoclaved acidified water and mouse chow ad libitum. All cells were purified from 8-1 2 week-old female mice; each purification involved pools of tissue from at least 4 mice, and each population was purified on two separate occasions and applied to independent arrays (biological replicates). RNA from all samples was processed together and amplified from approximately the same number of cells prior to hybridization to Affymetrix MOE430 2.0 microarrays. These arrays include ˜45,000 probe sets that, due to probe set redundancy, represent about two-thirds of the known coding genome. Each cell population was isolated from whole bone marrow (WBM), splenocytes, or peripheral blood (PB) of four mice. WBM was isolated by flushing the tibias and femurs with Hank's Balanced Salt Solution (HBSS; Invitrogen). Spleens were collected, placed in HBSS, crushed, and filtered in order to obtain splenocytes. PB was collected by retro-orbital bleeds and placed in 2% Dextran T500 in PBS with IOU of heparin. PB was allowed to sit for 30 minutes and the top layer was collected. Cells were then resuspended at 1×10⁸ cells/ml and stained on ice for 15 minutes with population-specific antibody cocktails followed by an HBSS wash. They were then isolated via a triple-laser instrument (MoFlow, Cytomation). All antibodies were obtained from BD Pharmigen unless otherwise stated.

Erythrocytes, granulocytes, and LT-HSCs were isolated from WBM. Nucleated erythrocytes were Ter-119⁺, CD3⁻, CD4⁻, CD8⁻, Mac-1⁻, Gr-1⁻, and B220⁻. Granulocytes were Gr-1⁺, clone 7/4⁺ (Cedarlane Labs), CD2⁻, CD5⁻, B220⁻, F4/80⁻ (eBiosciences), ICAM-I⁻, Ter-119⁻. LT-HSCs were isolated. Briefly, WBM was stained with Hoescht 33342 and the Sca-1⁺ cells were enriched by magnetic separation, followed by flow cytometry for sidepopulation (SP) and Sca-1⁺, c-it⁺, and Lin⁻ (Mac-1, Gr-1, Ter119, B220, CD4, CD8 (eBiosciences). T-cells and B-cells were isolated from splenocytes. Naive helper T-cells were freshly isolated CD⁺, CD25⁻, CD69⁻ cells. Naïve cytotoxic T-cells were CD4⁺, CD25⁻, and CD69⁻. Splenocytes enriched for naive T-cells were isolated and treated with concanavalin A (1 pg 1 ml, Sigma) to obtain activated T-cells. Eight to eleven hours after ConA stimulation, activated T-cells were isolated by sorting for the markers CD25⁺ and CD69 B⁺. B-cells were CD19⁺ and 33D1⁻ splenocytes. Monocytes were isolated from PB based on Forward and Side Scatter properties as well as being Mac-1⁺. NK cells were isolated from spleen, as Nk1.1⁺ and CD3⁻ cells.

### RNA Purification and Hybridization to Microarrays

RNA was isolated from 1×10⁵ cells (except for HSCs which was from 2.5-5×10⁴ cells), using the RNAqeuous kit (Ambion, Austin, Tex., USA), treated with DNAseI, and precipitated with phenol:chloroform:isoamyl alcohol. The RNA was linearly amplified as previously reported. Briefly, two rounds of T7-based in vitro transcription using the MessageAmp kit (Ambion) was undertaken and the RNA was labeled with biotin-conjugated UTP and CTP (Enzo Biotech). Amplified biotinylated RNA (20 ug) was diluted in fragmentation buffer, incubated at 94° C. for 25 minutes, and stored at −80° C. A sample was run on a 4% agarose gel to confirm an RNA fragment length of approximately 50 bp. The labeled RNA was hybridized to MOE430.2 chips according to standard protocols. For signal amplification, the chips were washed and counterstained with PE-conjugated strepavidin and a biotinylated anti-streptavidin antibody. The raw image and intensity files were generated using GCOS 1.0 software (Affymetrix). All microarrays used in this study had to pass several quality control tests including a scale factor<5, a 5′ to 3′ probe ratio<20, and replicate correlation coefficient>0.96.

### Microarray Analysis

Normalization and model-based expression measurements were performed with GC-RMA. GC-RMA, as well as additional analytical and annotation packages, are available as part of the open-source Bioconductor project (www.bioconductor.org) within the statistical programming language R (www.cran.r-project.org). Cluster and Principle Components Analyses were performed with the R base package. Lineage fingerprints were established by setting on/off expression thresholds (Off<4, On>5) and using Boolean logic to find uniquely expressed genes in each cell type and each grouping. When selecting a T-cell fingerprint, activated T-cells were removed from the data set and naïve T-cells (both CD4⁺ and CD8⁺) were averaged together.

### Knockout Analysis

All knockout data were obtained via the 3.44 MGI database (www.informatics.jax.org). Knockouts with a reported hematopoietic or immune phenotype were grouped into a single list (‘hematopoietic knockout data’). All genes on the array that have a reported knockout were identified. The frequency of fingerprint genes that have a hematopoietic phenotype was compared to the frequency of hematopoietic knockouts found by chance within all knock-out data. A Z-statistic was used to determine the significance of enrichment fingerprint genes found within the knockouts with a reported hematopoietic phenotype. A Z-statistic above 3 S.D. was considered significant for this enrichment.

### KEGG Analysis

KEGG is a suite of gene databases systematically organized into metabolic and signaling pathways (www.genome.ad.jp/kegg/). The mean expression value for each hematopoietic cell type was obtained for all KEGG pathways that contained genes found on the microarray. Pathways with a significant variation between the cell types (ANOVA p-value≦0.05) were identified (65 significant pathways). The pathways were then ranked by maximal abundance for each cell type and a heat map was generated using the centered data.

### Chromosomal Analysis

Affymetrix probes were assigned a value of 1 if they were equal to or greater than 4.5 and 0 if they were below 4.5. Each probe was then plotted to its chromosomal position and a chromosomal expression map was created for each chromosome for each cell type by an R function we developed. Briefly, this function uses a sliding and overlapping window, which covers 20 probe sets (representing 14 genes on average) at one time and overlaps with the previous nineteen, that takes the sum of 1's within the window for the y-axis and the mean chromosomal position for the x-axis and uses these values to plot the expression map. For comparison between cell types, the expression map of one cell type was directly subtracted from all the other cell types in turn. To quantitate openness of chromatin, the area under the curves was calculated and divided by the possible area under the curve (if all y-values were 20) to calculate a percentage of more open chromatin as shown by the chromosomal expression maps.

### Retroviral HSC Transduction

Clones for Ets2, Zfp105, Chd7 (chromodomain helicase DNA binding protein 7), Edaradd (EDAR (ectodysplasin-A receptor)-associated death domain), Med8 (mediator of RNA polymerase II transcription, subunit 8 homolog), Med14 (mediator complex subunit 14), Glis2 (GLIS family zinc finger 2), Dzip1 (DAZ interacting protein 1), Tnfaip8l1 (tumor necrosis factor, alpha-induced protein 8-like 1), 2210016F16Rik, Tbl1x Retroviral transduction of Tbl1x (transducin (beta)-like 1 X-linked), and Mina (myc induced nuclear antigen) were TA-cloned into a PENTR/D-TOPO vector and the Gateway system was used to recombine Ets2, Zfp105, Chd7, EDAR, Med8, Med14, Glis2, Dzip1, Tnfaip8l1, 2210016F16Rik, Tbl1x, and Mina into a murine stem cell virus (MSCV) vector containing attR recombination sites. MSCV was produced and packaged using 293T cells by cotransfecting MSCV target vectors with pCL-Eco packaging vector. CD45.2 mice were treated with 5-Fluorouracil (150 mg/kg American Pharmaceutical Partners) 6 days before harvesting bone marrow. Bone marrow was enriched for Sca-1⁺ cells using magnetic enrichment (AutoMACS, Miltenyi) and adjusted to a concentration of 5×10⁵ cells/ml in transduction medium, containing Stempro 34 (Gibco), nutrient supplement, penicillin/streptomycin, L-Glutamine (2 mM), mSCF (10 ng/ml, R&D Systems), mTPO (100 ng/ml, R&D Systems), and polybrene (4 ug/ml, Sigma). Thawed virus was applied at an MOI of 1 and the suspension was spin-infected at 250 g at room temperature for 2 hours. After transduction, the cells were incubated at 37° C. for 3 hours and transplanted into lethally irradiated CD45.1 mice. Engraftment, transduction and lineage analysis was performed on peripheral blood 12 weeks after transplantation by FACS (FACSaria, BD).

The results of the experiments presented in this Example are now described.

### Example 1

### Gene Expression Patterns Reflect Ontogeny

To identify genes uniquely expressed in HSC and their differentiated progeny, a global gene expression profiling approach was used to determine in parallel the transcribed genome of known coding transcripts in freshly isolated HSC as well as the major differentiated hematopoietic lineages, including natural killer (NK) cells, T-cells, B-cells, monocytes, neutrophils, and nucleated erythrocytes. Both activated and naive CD4+ (helper) and CD8+ (cytotoxic) T-cell subsets were examined. Each population was purified to at least 95% purity, and multiple parameters were standardized to reduce technical variation. RNA from the samples was processed and hybridized to Affymetrix MOE430 2.0 microarrays, which have probe sets representing about 20,000 genes (˜⅔ of the known coding genome). The correlation coefficients between chip pairs were at least 0.96, indicating very high data quality. This is the first study to interrogate a stem cell and multiple specific progeny types.

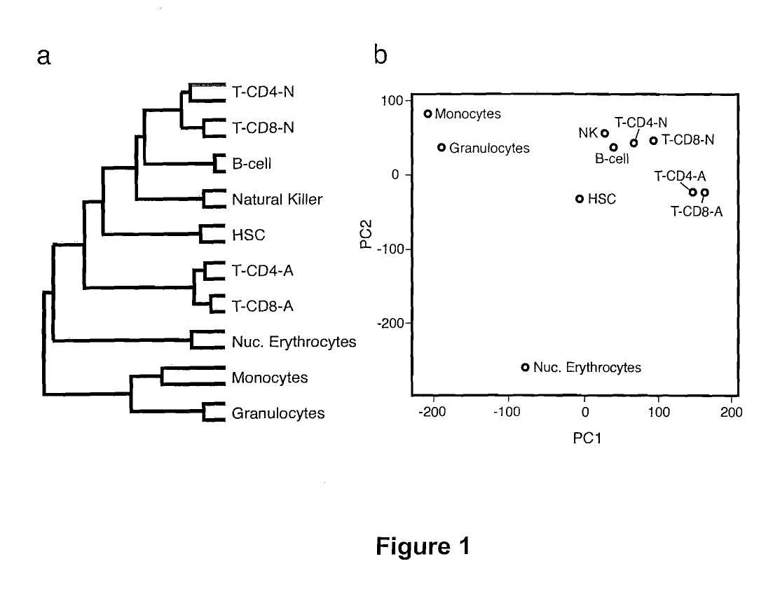

The data set was first used to investigate assumptions concerning relatedness of the hematopoietic cell types, as the similarity of gene expression profiles between different populations should reflect their ontogeny relationships, and perhaps shed light on the debate regarding the developmental origins of the erythroid branch. Cluster analysis was used to assess relative distance of the transcriptome of each cell type, resulting in a branched “family tree” based on the similarity between overall transcription patterns (FIG. 1A). HSC clustered with the lymphocytes, suggesting a strong similarity in their transcriptional activity. This observation supports a recent report that HSC and lymphocytes share many similarities, including cell surface activation molecules, and may indicate a conserved mechanism between HSC and lymphocytes of long term quiescence interrupted by bursts of proliferative stimuli. Also noteworthy is the very distinct nature of activated T-cells (both CD4+ and CD8+).

Principle components analysis (PCA) was used to further explore the cell-type relationships. When the relative expression distance between each averaged chip pair was examined with PCA in two dimensions (first and second principle components; PC1, PC2), the cell types cluster on the basis of ontogeny in the hematopoietic tree, with the stem cell having a centralized position (FIG. 1B). This analysis reveals the striking segregation of lymphoid, myeloid, and erythroid cells into three discrete clusters. Both cluster analysis and PCA indicate that erythrocytes are distant from myeloid cells (monocytes, granulocytes), supporting a recent observation that they arise independently of a common myeloid progenitor.

### Experiment #2: Genetic Fingerprints Unique to Each Cell Type

The data set was next examined to determine whether there were genes exclusively expressed by particular cell types. Such a genetic “fingerprint” would be expected to contain some genes encoding proteins involved in functions unique to that cell type, as well as regulatory proteins that may direct expression of other genes in the fingerprint.

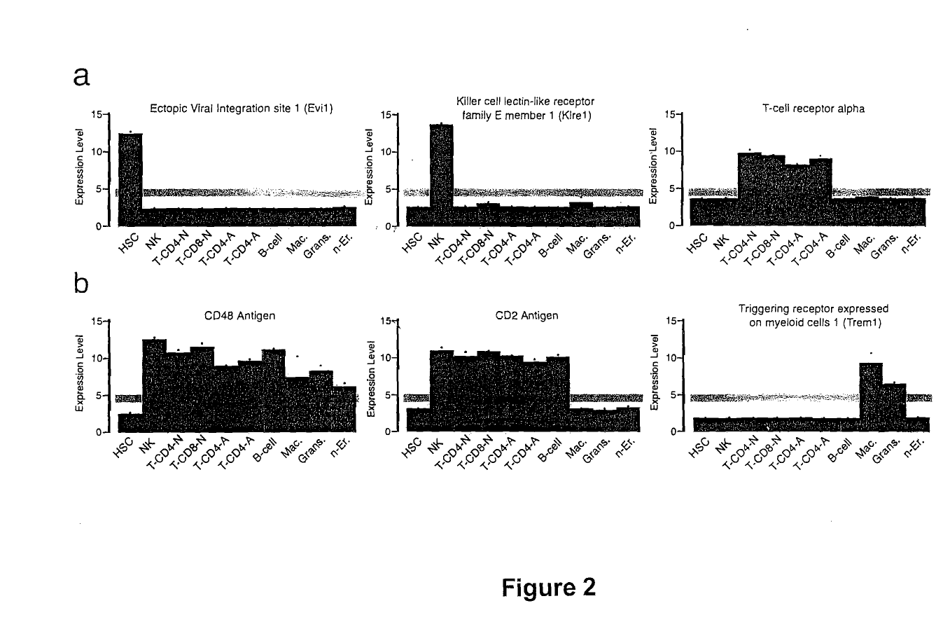

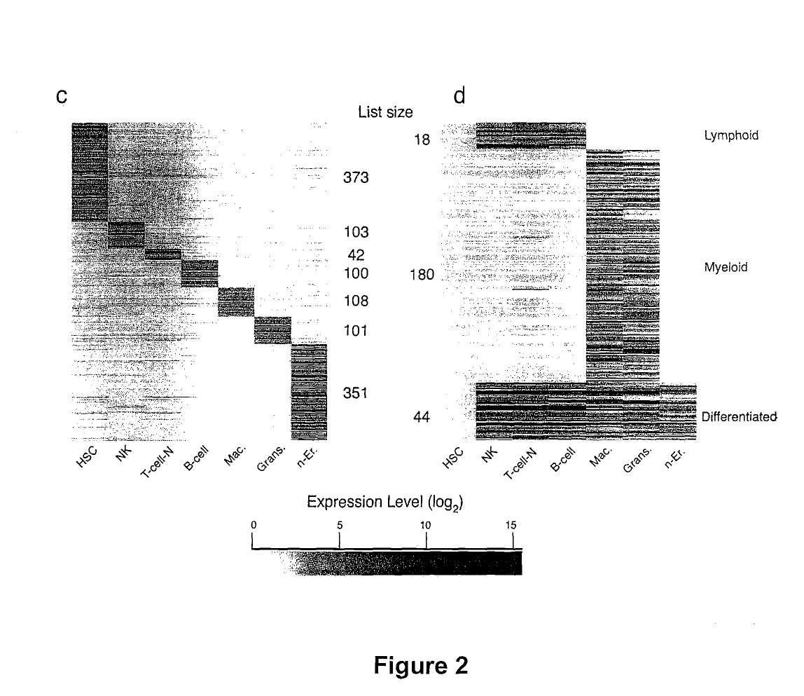

“Fingerprint genes,” as used herein, refer to those that were observed to be expressed in one cell type, but not expressed in all other profiles. Expression thresholds above and below a conservative window were selected by real-time PCR. An expression value of 5 or greater was considered to be expressed in that cell type, and 4 or less was considered to be not expressed in that cell type (log(2) scale). Using these thresholds to filter the data, unique genes expressed in each cell type were identified. Examples of genes found in fingerprints are ecotropic viral integration site-1 (Evi1; a known proto-oncogene), one of the most differentially expressed genes in HSC, killer cell lectin-like receptor family E member-1 (Klre1; a known NK cell receptor), and T cell receptor alpha on T cells (FIG. 2A).

The filter was also applied to groups of cell-types to determine a “shared” fingerprint for all differentiated cells, as well as the lymphoid (NK cells, T-cells, B-cells) and myeloid (monocytes and granulocytes) branches, which defined mutually expressed genes excluded from other cells (FIG. 2B). Strikingly, expression of the gene for the cell surface marker CD48 identifies all the differentiated cell types but not HSC, consistent with recent reports and antibody staining (not shown). Similarly, CD2 expression marks the lymphoid lineage, and Trem1 the myeloid lineage. Due to the similarities of naive CD4+ and CD8+ T-cells and their activated counterparts, for the majority of these analyses naive T-cells were used to represent the T-cell lineage. The relationships between the T-cell sub-types are dissected further below.

Each cell type fingerprint contained 40-100 genes, except the HSC and erythroid cells, which both had ˜350 genes (FIG. 2C). The smallest fingerprint was that of the T-cells, which had only 42 unique genes, due to the similarities between T-cells and other lymphoid cells. The NK and B-cell fingerprints were each around 100 genes. The shared lymphoid fingerprint, representing genes shared by all lymphoid cells and excluded from stem cells, contained only 18 genes, reflecting their similarity to stem cells. The myeloid fingerprint contained 154 genes, and the differentiated fingerprint, which includes all differentiated cell types and excludes stem cells, contains 36 genes (FIG. 2D). Finally, genes expressed in all cell types examined were also identified to establish a common hematopoietic signature of ˜9000 genes. Many of these are likely house-keeping genes shared with many non-hematopoietic cell types, but some are unique to the entire hematopoietic system, such as CD45.

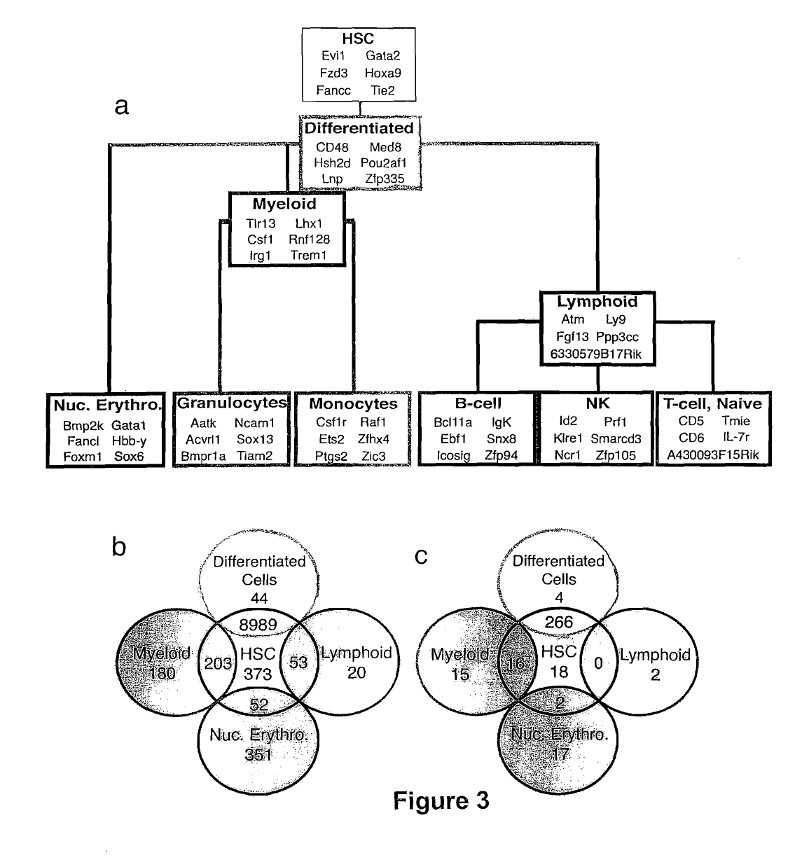

The genetic fingerprints contain some genes known to be important for the function of certain cell types, such as Gatal in the erythroid lineage and the CSF1 receptor (Csf1r) in the monocyte lineage, validating the approach for identification of cell-type-specific genes (FIG. 3A). The fingerprints also contain many under-studied genes that may have regulatory role or may serve as novel markers for those cell types. Likewise, the shared fingerprint genes are either required for the function of all family cell types, such as Atm in the lymphoid fingerprint, or for differentiation of the cell-type family (FIG. 3A). The progenitors of differentiated branches are likely to express some of the shared fingerprint genes (FIG. 3A and FIG. 3B), which could be developed as tools for markers of those progenitors, or as regulators of their fate.

To assess whether deficiencies of fingerprint genes result in hematopoietic cell defects, reported phenotypes in the Jax Mouse Genome Informatics database (v.3.44) were examined for all genes that had a reported knock-out allele. Remarkably, several of the fingerprint genes with available knock-outs show serious defects in either differentiation or functionality of a lineage (FIG. 3C, Table 1). A striking example from the B-cell fingerprint is the Ebf1 knockout mouse, which exhibits a complete loss of B-cells. Likewise, ablation of HoxA9, an HSC fingerprint gene, results in severely impaired HSC. All fingerprints, with one exception, demonstrated a statistically significant enrichment of genes exhibiting a defect in development or function of hematopoietic cells when knocked-out (z-test). Only the whole common fingerprint, which contains all genes expressed in common in all ten populations, was not found to be enriched for a hematopoietic knock-out phenotype, most likely due to the preponderance of house-keeping genes within this list.

TABLE 1

| Fingerprint | Gene Name | Gene Symbol | Knockout Phenotypes from MGI |
|---|---|---|---|
| HSC | GATA binding protein 2 | Gata2 | Abnormal hematopoiesis |
| | Myeloproliferative leukemia | Mpl | Abnormal hematopoiesis |
| | virus oncogene | | |
| | Endoglin | Eng | Abnormal hematopoiesis |
| | Endothelial-specific receptor | Tek | Abnormal hematopoiesis |
| | tyrosine kinase | | |
| | Neuroblastoma myc-related | Nmyc1 | Embryonic lethality |
| | oncogene 1 | | |
| | Ecotropic viral integration site | Evi1 | Embryonic lethality |
| | 1 | | |
| B-cells | Early B-cell factor 1 | Ebf1 | B-cells fail to mature |
| | Immunoglobulin heavy chain | Lghg | NA |
| | (gamma polypeptide) | | |
| | CD79A antigen | Cd79a | Arrested B-cell |
| | (immunoglobulin-associated | | development and decreased |
| | alpha) | | B-cell number |
| | CD37 antigen | Cd37 | Decreased IgG, abnormal |
| | | | B-cell response |
| | B-cell CLL/lymphoma 11A | Bcl11a | B-cell deficiency |
| | (zinc finger protein) | | |
| T-cells, naïve | Transmembrane inner ear | Tmie | Not examined |
| | IL2-inducible T-cell kinase | Itk | Decreased T-cell |
| | | | proliferation |
| | Interleukin 7 receptor | Il7r | Abnormal T-cell |
| | | | development, decreased T- |
| | | | cells |
| | T-cell receptor interacting | Tcrim | NA |
| | molecule | | |
| | T-cell receptor alpha chain | Tcra | Abnormal T-cell |
| | | | development |
| NK cells | Zinc finger protein 105 | Zfp105 | Na |
| | Killer cell lectin-like receptor | Klre1 | Defective NK cell cytolysis |
| | family E member 1 | | |
| | Fas ligand (TNF superfamily, | Fas1 | Increased T-cell and |
| | member 6) | | neutrophil number |
| | Killer cell lectin-like receptor, | Klra4 | Chinese hamster ovary |
| | subfamily A, member 4 | | killing allele |
| | Killer cell lectin-like receptor | Klrb1c | NA |
| | subfamily B member 1C | | |
| Granulocytes | SRY-box containing gene 13 | Sox13 | NA |
| | Cytochrome b-245, beta | Cybb | Dysregulated inflammatory |
| | polypeptide | | response |
| | Neural adhesion molecule 1 | Ncam1 | Reduced brain size |
| | Prostaglandin-endoperoxide | Ptgs1 | Decreased inflammatory |
| | synthase 1 | | response |
| | Apoptosis-associated tyrosine | Aatk | NA |
| | kinase | | |
| Monocytes | E26 avian leukemia oncogene | Ets2 | Absent vitelline blood |
| | 2,3′ domain | | vessels |
| | Prostaglandin-endoperoxide | Ptgs2 | Myeloid hyperplasia in |
| | synthase 2 | | spleen |
| | Colony stimulating factor 1 | Csf1r | Mononuclear phagocyte |
| | receptor | | deficiency |
| | Macrophage scavenger | Msr1 | Decreased ability to fight |
| | receptor 1 | | infection |
| | Interleukin 1 receptor | Il1rap | Required for IL-1 signaling |
| | accessory protein | | |
| Erythrocytes | Gata binding protein 1 | Gata1 | Abnormal erythropoiesis |
| | | | and anemia |
| | Pyruvate kinase liver and red | Pklr | Hemolytic anemia |
| | blood cell | | |
| | Intercellular adhesion molecule | Icam4 | NA |
| | 4 | | |
| | Acetylcholinesterase | Ache | Postnatal lethality |
| | Glial fibrillary acidic protein | Gfap | Increased susceptibility to |
| | | | injury |
| Lineage | Zinc finger protein, subfamily | Zfpn1a3 | Abnormal lymphocyte |
| | 1A, 3 (Aiolos) | | proliferation, candidate for |
| | | | lupus |
| | CD48 antigen | Cd48 | Abnormal T-cell |
| | | | development and |
| | | | proliferation |
| | POU domain, class2, | Pou2af1 | Abnormal B-cell |
| | associating factor 1 | | development |
| | Protein phosphatase 1, | Ppp1r10 | NA |
| | regulatory subunit 10 | | |
| | Leukocyte Ig-like receptor, | Lilrb4 | Abnormal hematopoieses |
| | subfamily B, member 4 | | and immune response |
| Myeloid | Toll-like receptor 4 | Tlr4 | Increased susceptibility to |
| | | | bacterial infection |
| | Pre B-cell leukemia | Pbx1 | Embryonic lethality from |
| | transcription factor 1 | | anemia and fewer myeloid |
| | | | progenitors |
| | Bone marrow stromal cell | Bst1 | NA |
| | antigen 1 | | |
| | Leukocyte receptor cluster | Leng4 | NA |
| | (LRC) member 4 | | |
| | Triggering receptor expressed | Trem1 | NA |
| | on myeloid cells 1 | | |
| Lymphoid | CD2 antigen | Cd2 | No phenotype reported |
| | Lymphocyte antigen 9 | Ly9 | NA |
| | Dual specificity phosphatase | Dusp19 | Not examined |
| | 19 | | |
| | Fibroblast growth factor 13 | Fgf13 | NA |
| | Plasmacytoma variant | Pvt1 | NA |
| | translocation 1 | | |

Lineage fingerprint genes: representational fingerprint genes are listed by gene name and Entrez Gene symbol for each fingerprint. Knock-out phenotypes are included where applicable according to Mouse Genome Informatics version 3.44. Key: “lineage” fingerprint: genes expressed in all cell types except the HSC; “Myeloid” fingerprint: genes expressed in both monocytes and granulocytes, but not other cell types; “Lymphoid” fingerprint: genes expressed in NK-, T-(all subsets), and B-cells, but not other cell types; “NA”: represents those genes where no knock out has been reported; “No phenotype reported”: genes where hematopoiesis was examined and no phenotype was found.

### Experiment #3: HSC and T-Cells Share a Similar Transcriptional Program of Activation.

Observations have suggested that HSC and T-cells share a number of markers as well as regulatory genes. These similarities could result from a similar life cycle of long-term quiescence followed by expansion, or this could be related to the evolutionary history of the hematopoietic system.

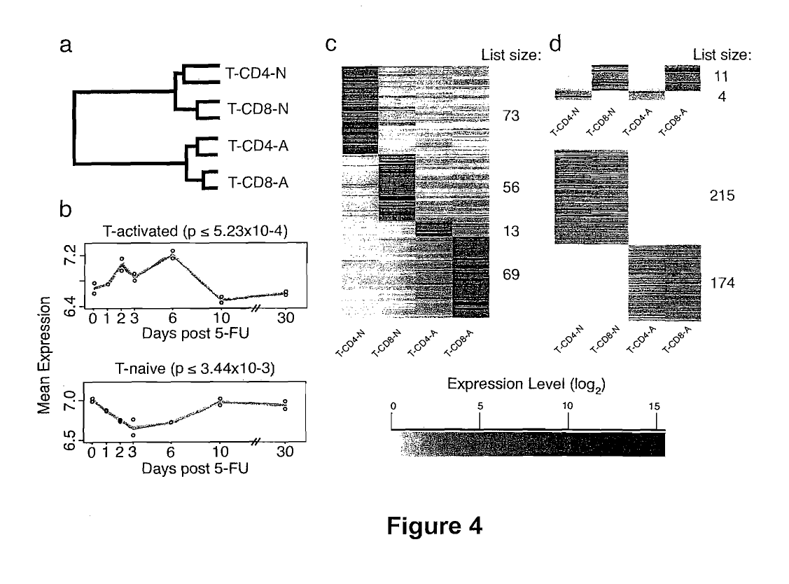

Cluster analysis was repeated with T-cell data alone. This analysis again showed that the activation state is far more important to distinguish T-cell sub-types than their CD4/CD8 status as helper or effector T-cells (FIG. 4A). A comparison of the activated and naive T-cell gene expression data to previous study in which genes were identified that change with HSC activation, as stimulated by 5-fluorouracil treatment, identified genes that differ between activated and naive T-cells, generated from a pair-wise comparison. The expression pattern of these genes in HSC over the activation time-course was examined, and the average expression values plotted. On average, genes that were upregulated in activated T-cells also showed a pattern of significant upregulation in activated HSC (FIG. 4B; genes increasing in expression on days 2-6 of HSC activation; one-way ANOVA p≦5.23×10⁴, alpha=0.05), whereas naive T-cell genes have the reciprocal signature, being more highly expressed in quiescent HSC (p≦3.44×10⁻³, alpha=0.05). Thus, quiescent HSC are more similar to naive T-cells, and proliferating HSC more similar to activated T-cells suggesting that the mechanisms that regulate the activation state in HSC and T-cells are similar.

Genes distinguishing the T-cell subsets were next identified, irrespective of their expression pattern in the other hematopoietic cells, using the same threshold criteria applied to the hematopoietic fingerprints. For each T-cell type, the number of genes uniquely expressed in each ranged from 13 (CD4-activated) to 73 (CD4-naive) (FIG. 4C). This strategy also revealed that the number of genes shared between activated and naive CD4+ T-cells, but excluded from their CD8+ counterparts, was only four; included is Zbtb7b, a gene that permits selection of CD4+ T-cells in the thymus. Activated and naive CD8+ cells shared merely eleven genes, including both the α and β chains of CD8 (FIG. 4D). However, the CD4+ and CD8+ naive T-cells shared 215 genes with each other, while the activated cells shared 174, consistent with the clustering behavior of these sub-types, and demonstrating the dramatic changes both helper and effector T-cells undergo during activation (FIG. 4A). As expected, activated T-cells upregulated established activation markers such as I12ra (CD2 5) and Icos. This list includes a substantial number of under-characterized genes, such as two Riken clones confirmed by RT-PCR (3110082i17Rik and 4933403f05Rik) named Heist (for Higher Expression in Stimulated T-cells) -1 and -2.

### Experiment #4: Genetic Pathway Analysis Reveals Key Pathways in Hematopoietic Cell Function.

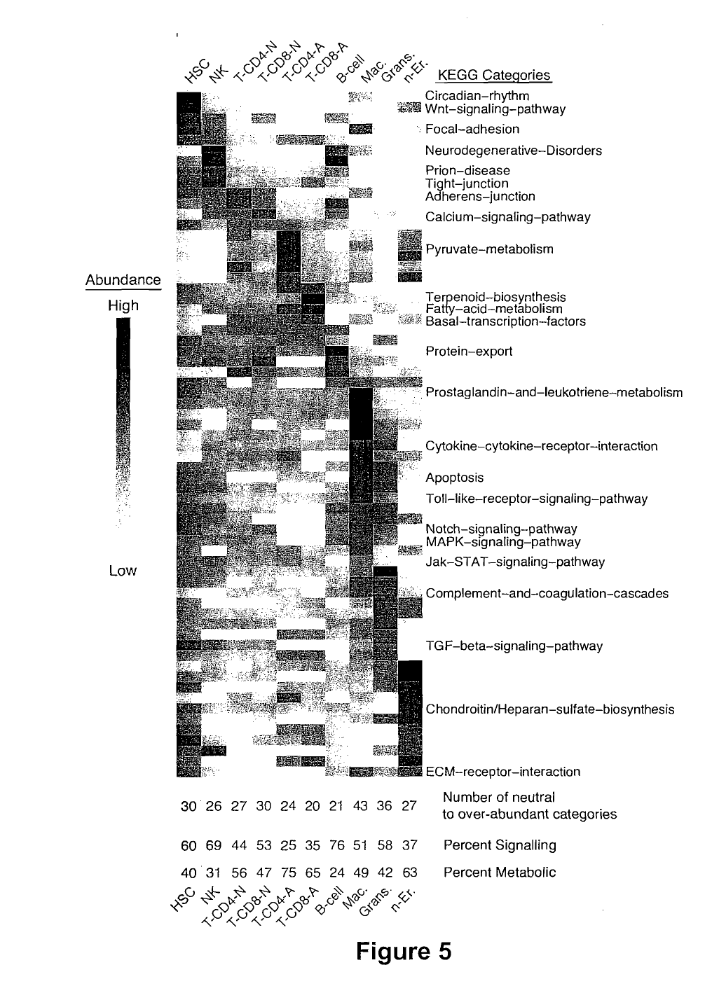

The Kyoto Encyclopedia of Genes and Genomes (KEGG) was used to group genes into specific pathways in order to investigate whether certain signaling or metabolic pathways are likely to be operating in specific cell types. The mean expression value for all arrayed genes in each pathway was determined for every cell type, and categories with significant variation (i.e. difference between the cell types; 65 categories; ANOVA p-value≦0.05, alpha=0.05) were displayed (FIG. 5). Notably, genes involved in Wnt signaling were over-represented in HSC, consistent with a putative role for this pathway in cell fate decisions. In addition, prostaglandin and leukotriene metabolism was found enriched in myeloid cells, and Notch signaling enriched in HSC, activated CD8+ T-cells, and monocytes.

Myeloid cells displayed the greatest over-abundance of both active metabolic and signaling pathways, with similar numbers of each pathway type being up regulated. T-cells showed a pattern of increasing metabolic pathways and decreasing signaling pathways upon activation. B-cells showed a 3-fold greater enrichment of signaling pathways over metabolic pathways, and NK cells exhibited a similar skewing, perhaps indicative of their role as primary sensors of the immune system. Nucleated erythrocytes displayed lower abundance of a number of signaling pathways while at the same time exhibiting a greater proportion of metabolic pathways, as would be expected during the final stages erythrocyte differentiation represented in the purified cells. HSC showed the third highest abundance of active pathways with signaling pathways represented more highly than metabolic pathways, consistent with a state of receptive readiness in the quiescent HSC, poised to differentiate into mature blood cell types.

Experiment #5: Bioinformatic Analysis Indicates HSC have an Open Chromatin State.

Stem cells have been speculated to maintain an open chromatin state, in order to facilitate rapid access to multiple lineage differentiation programs, with chromatin becoming more closed as differentiation occur. This notion reflects an idea of stem cell ‘priming” where multipotent progenitors may express low levels of genes necessary for multiple lineage programs, and shut down expression as lineage choices become restricted during differentiation, and suggests a major role for chromatin status in maintenance of “stemness” and chromatin remodeling in differentiation.

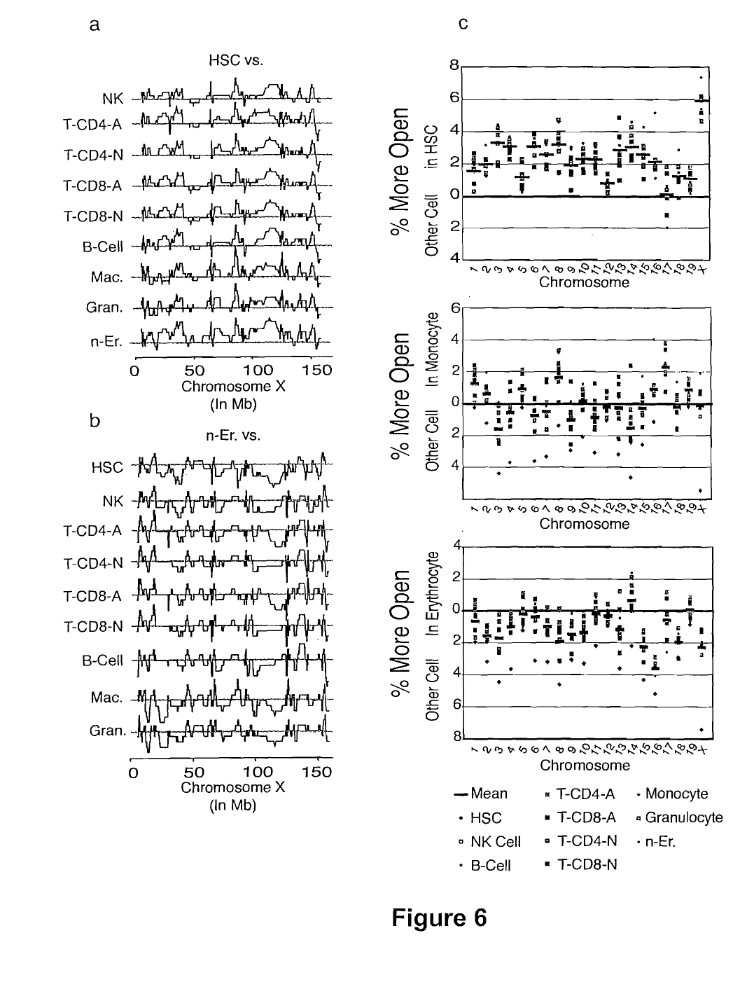

The small numbers of HSC that typically can be obtained prevent direct examination of the chromatin state using traditional methods. Thus, chromatin status was probed using high-level analysis of gene expression data by reasoning that “open” chromatin would translate into measurable expression by cohorts of physically adjacent genes along chromosomes. An algorithm was created to generate a map, for each cell type, of all transcribed genes (using 4.5 as the expression threshold), and to identify genes within windows of at least 20 adjacent co-expressed probe sets (representing 14 genes on average). Each cell type's chromosomal expression map was subtracted from the chromosomal expression maps of each other population to observe differences in chromosomal transcribed regions, as illustrated in FIG. 6A. When HSC were compared to all other cell types across the X-chromosome, the HSC exhibited many more regions of open chromatin, evidenced by peaks on the plots. The reverse is true for the erythrocytes (FIG. 6B), with mostly valleys. By calculating the area under the curve, the percentage of open chromatin for each cell type was determined and displayed graphically for each chromosome (FIG. 6C). On all chromosomes except 17, the chromatin is highly accessible in HSC, relative to the other cell types. Remarkably, the X-chromosome displays an extreme degree of openness. The same analysis with monocytes shows a variable degree of chromatin accessibility, while erythrocytes are largely closed except for chromosome 14. The marked openness of chromosome 14 in erythrocytes may indicate the presence of a number of linked genes involved in erythrocyte generation, although it is not clear what these might be.

Experiment #6: Fingerprint Transcription Factors can Bias Differentiation to their Lineage

As shown above, each cell-type-specific fingerprint contains a number of genes, some of which are likely to be involved in lineage specification. To test this, the ability of overexpression of putative regulatory fingerprint transcription factors to bias hematopoietic differentiation was examined. Two transcription factors were tested: Ets2, from the monocyte fingerprint representing the myeloid branch, and zinc finger protein 105 (Zfp105), from the NK cell fingerprint representing the lymphoid branch.

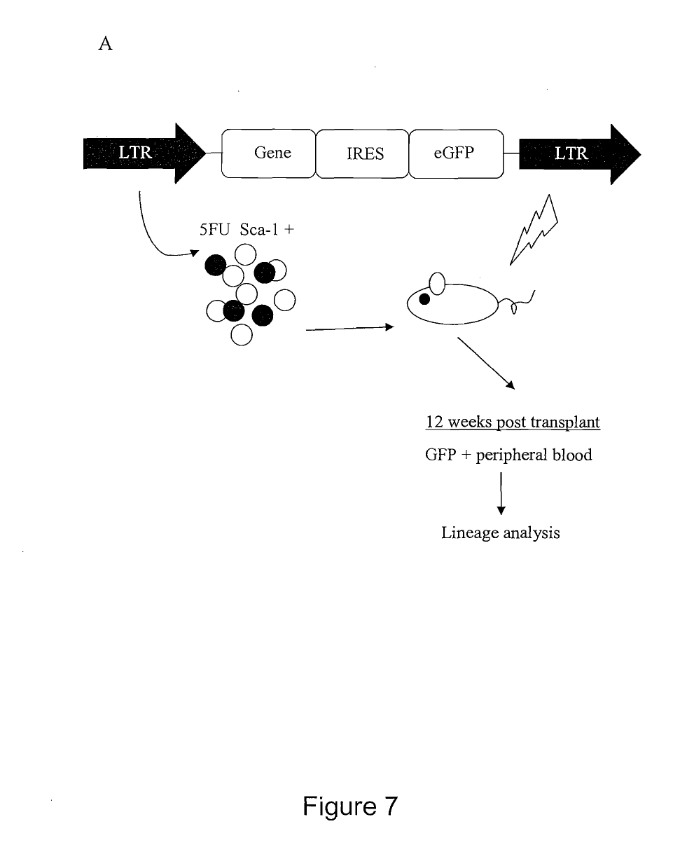

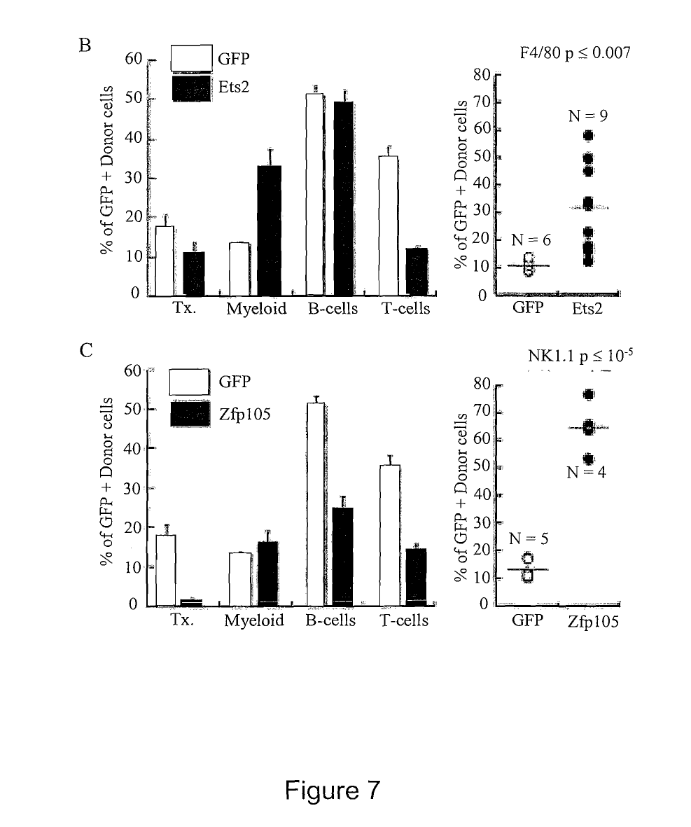

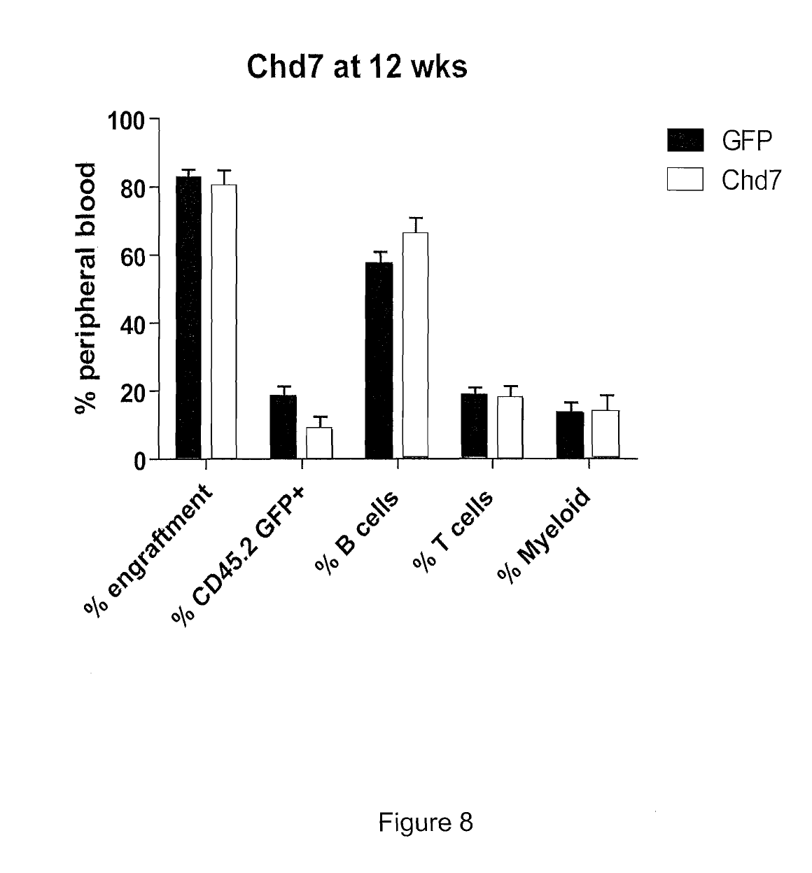

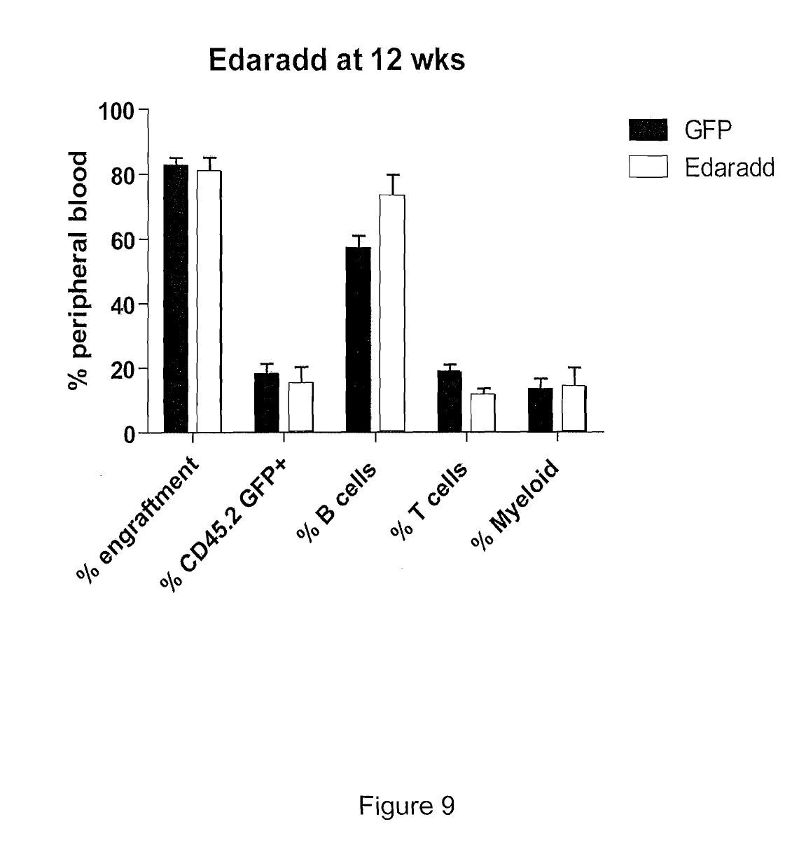

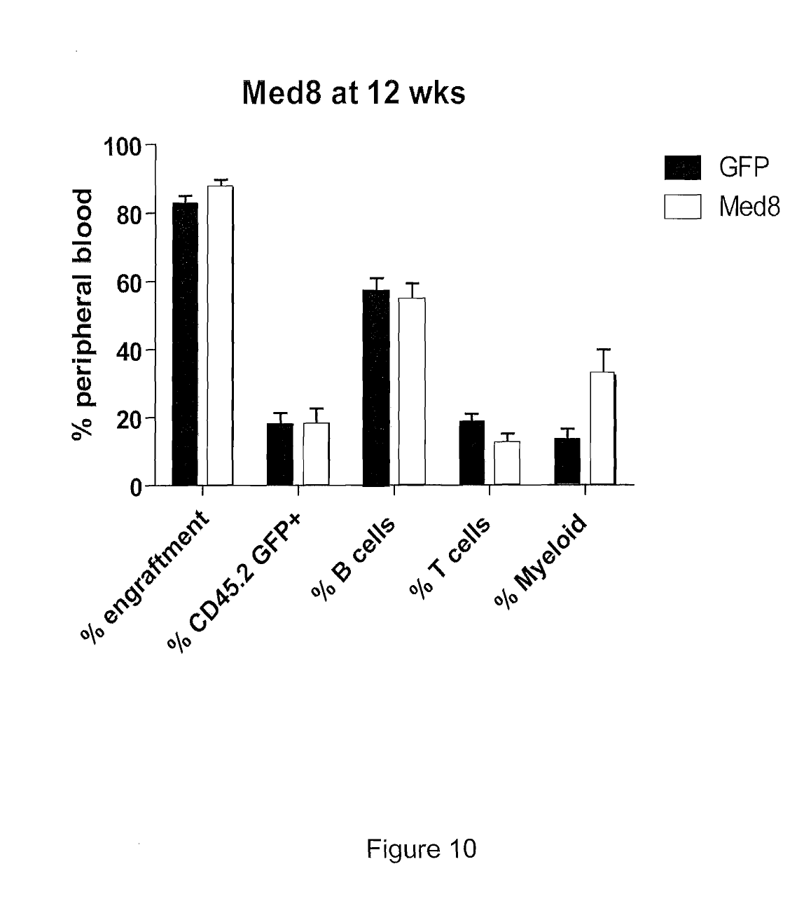

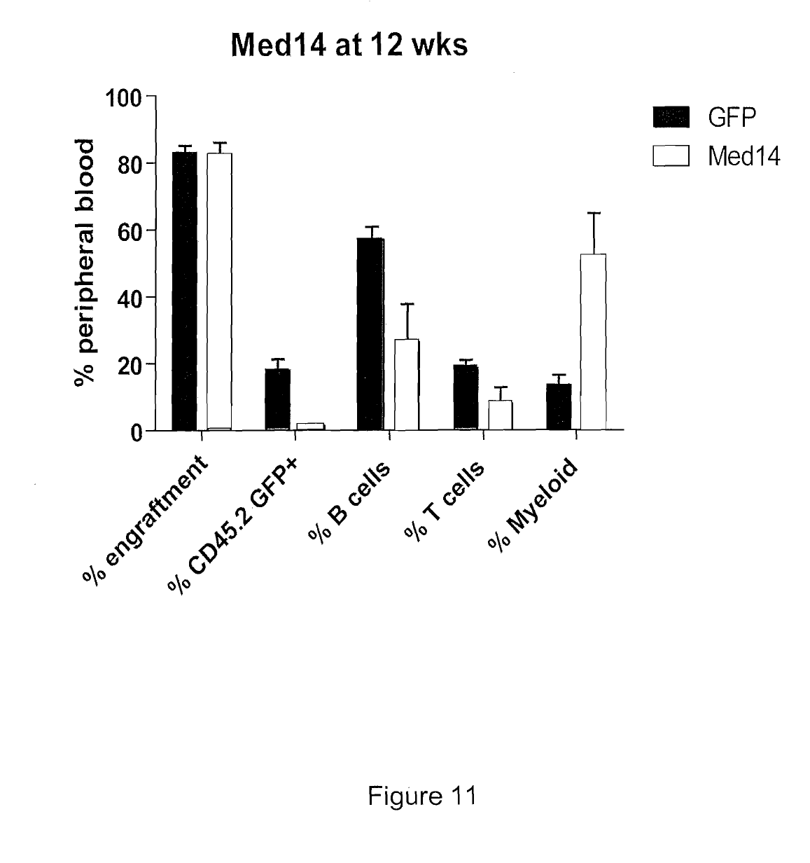

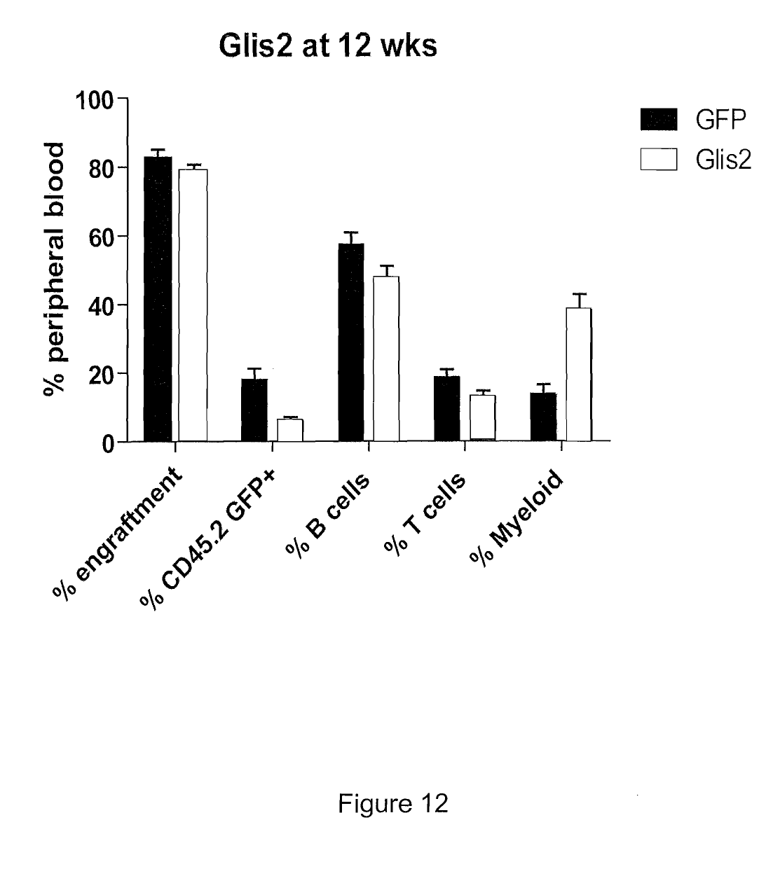

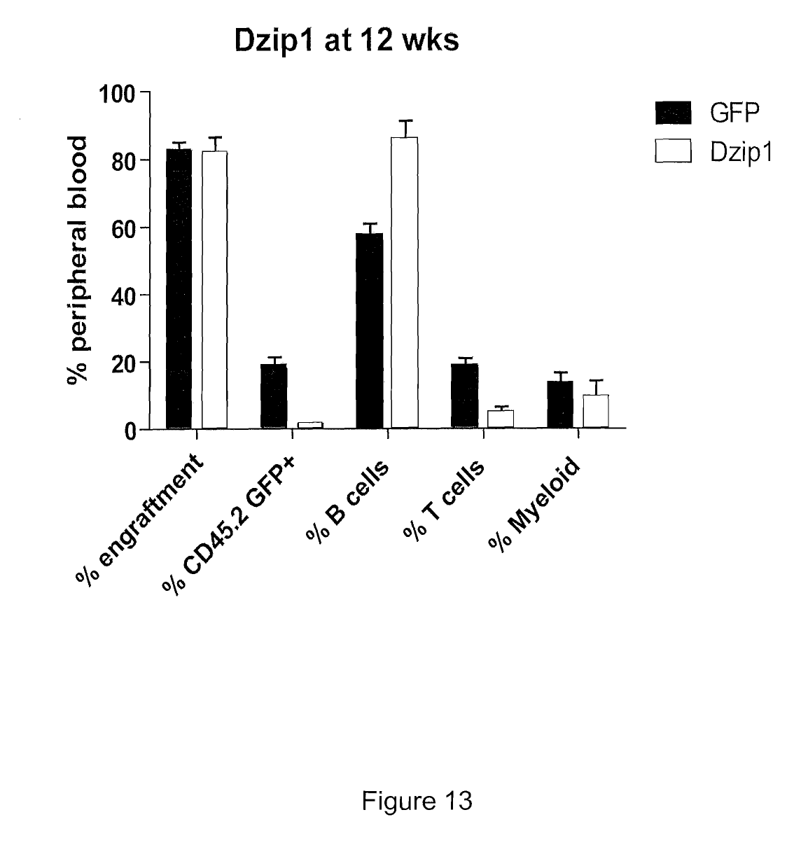

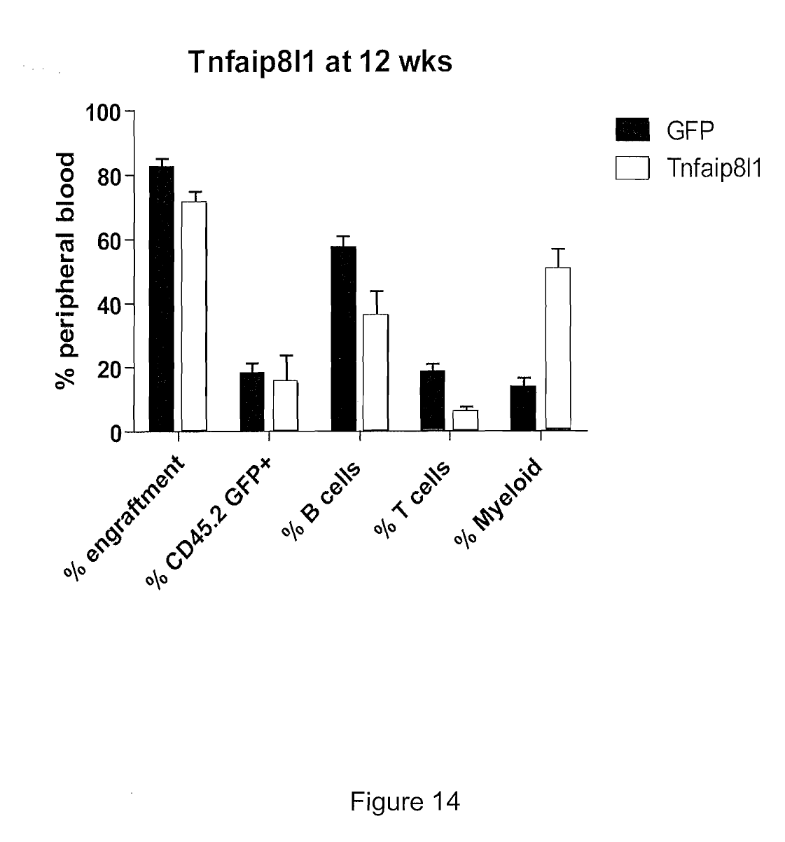

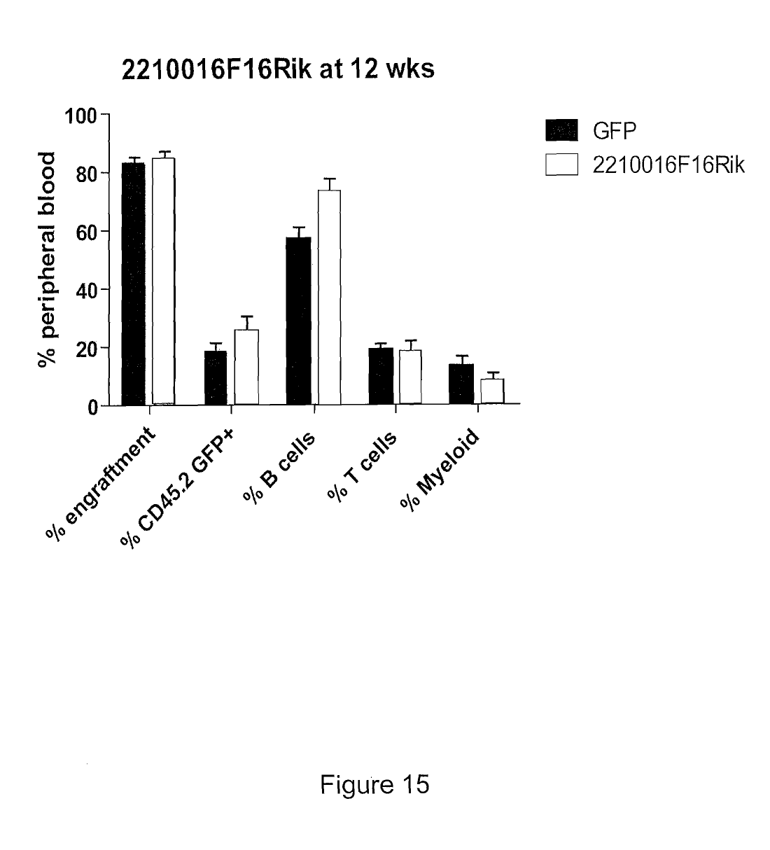

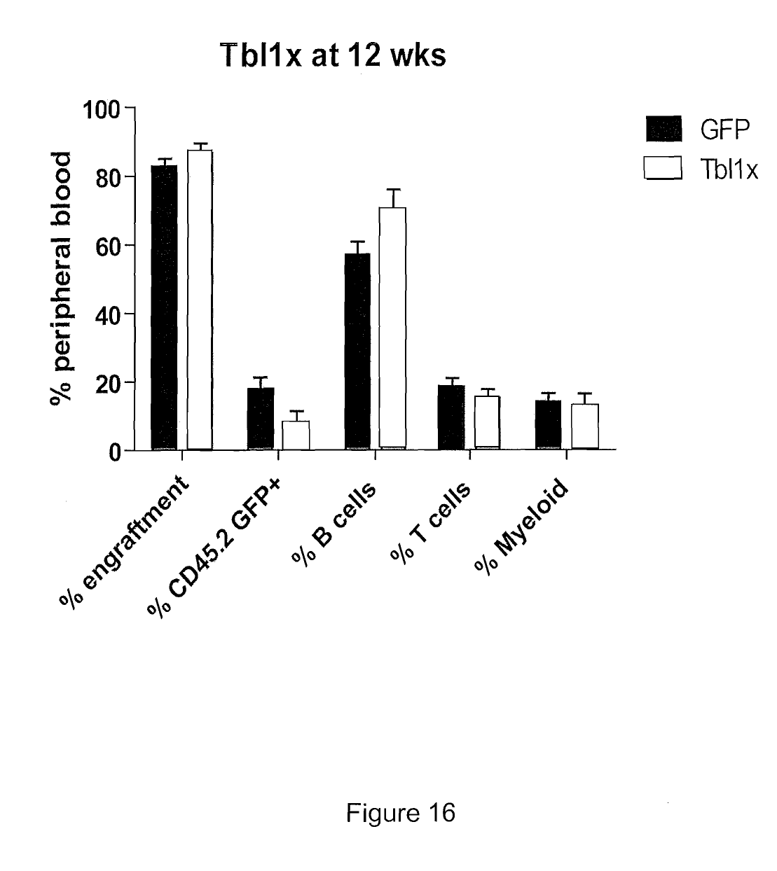

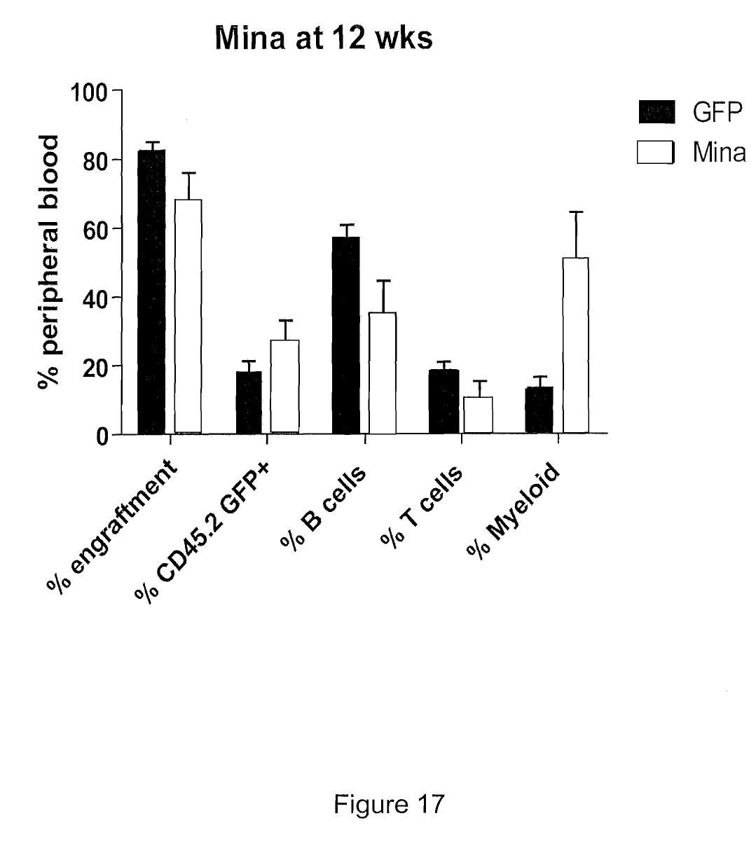

MSCV-based retroviral vectors containing each of the genes coupled to eGFP via an IRES were used to transduce bone marrow progenitors, which were then transplanted into lethally irradiated recipient mice (FIG. 7A). Bone marrow transduced with a vector containing eGFP alone served as a control. Peripheral blood of the recipients was examined 12 weeks after transplantation for the proportion of transduced GFP+ cells in different blood lineages, and lineage contribution in control vs. tester mice was compared. Ets2 overexpression resulted in an overall increase in myeloid cells, and a substantial decrease in T-cells relative to controls (FIG. 7b). When the F4/80 marker was used to specifically track monocyte differentiation, a ˜4-fold increase in monocytes derived from Ets2-transduced stem cells was evident in the peripheral blood, relative to controls (two sample T-test assuming equal variances, two sided p≦0.00 7, alpha=0.05), indicating that overexpression of Ets2 bias differentiation toward the macrophage lineage. Overall, both T-cells and B-cells were significantly reduced in the Zfp105 overexpressing blood cells relative to controls (FIG. 7C). When the proportion of NK cells in these mice was examined using the NK-specific marker NK1.1, a greater than 5-fold increase in NK cells was observed (two sample T-test assuming equal variances, two-sided p≦10⁻⁶, alpha=0.05). Interestingly, the total percentage of Zfp105-transduced cells was quite low in these mice (˜2% at 12 weeks, compared to ˜18% in GFP-alone). This indicates that over expression of a powerful differentiation factor is incompatible with maintenance of stem cell functions, as required for multilineage contribution in this in vivo assay.

Retroviral transduction of Chd7 (chromodomain helicase DNA binding protein 7), a B-cell fingerprint gene, skews differentiation toward the B-cell lineage. Chd7 is a putative transcription factor associated with CHARGE syndrome in humans. Heterozygous Chd7 knock-out mice display the symptoms of CHARGE syndrome.

Retroviral transduction of Edaradd (EDAR (ectodysplasin-A receptor)-associated death domain), a B-cell fingerprint gene, skews differentiation toward the B-cell lineage. Edaradd is a cytoplasmic receptor previously implicated in fertility and hair follicle development in mice.

Retroviral transduction of Med8 (mediator of RNA polymerase II transcription, subunit 8 homolog), a Differentiated fingerprint gene, skews differentiation toward the myeloid lineages. Myeloid skewing is consistent with a role in stem cell expansion. Med8 is a putative transcription factor that may be part of the mediator complex.

Retroviral transduction of Med14 (mediator complex subunit 14), an HSC fingerprint gene, may induce quiescence, as seen by the low percentage of GFP+ cells at all time points. It also skews differentiation toward the myeloid lineages. Med14 is a putative transcription factor that may be part of the mediator complex.

Retroviral transduction of Glis2 (GLIS family zinc finger 2), an HSC fingerprint gene, skews differentiation toward the myeloid lineages. Glis2 is a transcription factor known to negatively regulate transcription. Myeloid skewing is consistent with a role in stem cell expansion. Homozygous Glis2 knock-outs exhibit kidney atrophy and cysts.

Retroviral transduction of Dzip1 (DAZ interacting protein 1), a naïve T-cell fingerprint gene, skews differentiation toward the B-cell lineage. Dzip1 has putative nucleic acid binding capacity and therefore is classified as a potential transcription factor.

Retroviral transduction of Tnfaip8l1 (tumor necrosis factor, alpha-induced protein 8-like 1), a naïve T-cell fingerprint gene, skews differentiation toward the myeloid lineages.

Retroviral transduction of 2210016F16Rik, an activated T-cell fingerprint gene, skews differentiation toward the B-cell lineage. 2210016F16Rik has no known function and belongs to a group of genes (Riken genes) identified by large scale sequencing of messenger RNA.

Retroviral transduction of Tbl1x (transducin (beta)-like 1 X-linked), an activated T-cell fingerprint gene, skews differentiation toward the B-cell lineage. Tbl1x is a transcription factor involved in nuclear receptor-mediated transcription.

Retroviral transduction of Mina (myc induced nuclear antigen), an activated T-cell fingerprint gene, skews differentiation toward the myeloid lineages. Mina is localized to the nucleus and may play a role in cellular proliferation.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of directing the differentiation of a hematopoietic stem cell (HSC), a hematopoietic progenitor cell (HPC), or a combination thereof, said method comprising introducing into said cell a zinc finger protein (Zfp105), wherein said Zfp105 upregulates expression of a natural killer (NK) cell-specific polynucleotide in said cell, thereby inducing differentiation of said cell into an NK cell.

2. The method of claim 1, wherein said Zfp105 is delivered to said cell as a polypeptide.

3. The method of claim 1, wherein said Zfp105 is delivered to said cell as a nucleic acid.

4. The method of claim 3, wherein said nucleic acid is contained within a vector.

5. The method of claim 4, wherein said vector is a viral vector.

6. The method of claim 5, wherein said viral vector is selected from the group consisting of a retroviral vector, an adenoviral vector, and an adeno-associated viral vector.

7. The method of claim 4, wherein said vector is a non-viral vector.

8. The method of claim 1, wherein said cell is human.

9. A method of directing the differentiation of a HSC, a HPC, or a combination thereof, said method comprising introducing into said cell an Ets2, wherein said Ets2 upregulates expression of a monocyte-specific polynucleotide in said cell thereby inducing differentiation of said cell into a monocyte.

10. The method of claim 9, wherein said Ets2 is delivered to said cell as a polypeptide.

11. The method of claim 9, wherein said Ets2 is delivered to said cell as a nucleic acid.

12. The method of claim 11, wherein said nucleic acid is contained within a vector.

13. The method of claim 12, wherein said vector is a viral vector.

14. The method of claim 13, wherein said viral vector is selected from the group consisting of a retroviral vector, an adenoviral vector, and an adeno-associated viral vector.

15. The method of claim 12, wherein said vector is a non-viral vector.

16. The method of claim 9, wherein said cell is human.

17. A method of directing the differentiation of HSC, a HPC, or a combination thereof, to a B-cell, said method comprising introducing to said cell at least one biomarker selected from the list consisting of Chd7 (chromodomain helicase DNA binding protein 7), Edaradd (EDAR (ectodysplasin-A receptor)-associated death domain, 2210016F16Rik, Dzip1 (DAZ interacting protein 1), and Tbl1x (transducin (beta)-like 1 X-linked).

18. The method of claim 17, wherein said biomarker is delivered to said cell as a polypeptide.

19. The method of claim 17, wherein said biomarker is delivered to said cell as a nucleic acid.

20. The method of claim 19, wherein said nucleic acid is contained within a vector.

21. The method of claim 20, wherein said vector is a viral vector.

22. The method of claim 21, wherein said viral vector is selected from the group consisting of a retroviral vector, an adenoviral vector, and an adeno-associated viral vector.

23. The method of claim 20, wherein said vector is a non-viral vector.

24. The method of claim 17, wherein said cell is human.

25. A method of directing the differentiation of a HSC, a HPC, or a combination thereof, to a cell of the myeloid lineage, said method comprising introducing to said cell at least one biomarker selected from the list consisting of Med8 (mediator of RNA polymerase II transcription, subunit 8 homolog), Med14 (mediator complex subunit 14), GLIS2 (GLIS family zinc finger 2), Tnfaip8l1 (tumor necrosis factor, alpha-induced protein 8-like 1), and Mina (myc induced nuclear antigen).

26. The method of claim 25, wherein said biomarker is delivered to said cell as a polypeptide.

27. The method of claim 25, wherein said biomarker is delivered to said cell as a nucleic acid.

28. The method of claim 27, wherein said nucleic acid is contained within a vector.

29. The method of claim 28, wherein said vector is a viral vector.

30. The method of claim 29, wherein said viral vector is selected from the group consisting of a retroviral vector, an adenoviral vector, and an adeno-associated viral vector.

31. The method of claim 28, wherein said vector is a non-viral vector.

32. The method of claim 25, wherein said cell is human.
